(19) United States
(12) Patent Application Publication
(10) Pub. No.: US 2026/0241132 A1
(43) Pub. Date: Aug. 20, 2026

(54) HEADGEAR FOR PATIENT INTERFACES

(71) Applicants: ResMed Pty Ltd, Bella Vista, New South Wales (AU); ResMed Asia Pte. Ltd., Singapore (SG)

(72) Inventors: Christopher Daniel PARKER, Sydney (AU); Adrian Jeffrey LOWRY, Singapore (SG); Lemmy NGA, Sydney (AU); Andrew James BATE, Sydney (AU); Shannon William PRIOR, Sydney (AU); Elisa Anne JEFFERY, Sydney (AU); Paul Derrick WATSON, Sydney (AU); Daniel Marc WEISS, Sydney (AU); Tyran Daniel LECHNER, Sydney (AU)

(21) Appl. No.: 19/471,913

(22) PCT Filed: Apr. 3, 2024
(86) PCT No.: PCT/AU2024/050316

§ 371 Date: Oct. 2, 2025

(30) Foreign Application Priority Data
Apr. 4, 2023 (AU) 2023900972
May 29, 2023 (AU) 2023901670

(51) Int. Cl. A61M 16/06

(52) U.S. Cl. CPC A61M 16/0683

(57) ABSTRACT

Disclosed are positioning and stabilising structures for supporting a patient interface in sealing engagement with the face of a patient during delivery of a flow of breathable gas to the nose and mouth of the patient during a respiratory therapy treatment. In some examples the positioning and stabilising structures are configured to extend above the ears of the patient, and may comprise rigidisers which extend from an anterior side of the patient's ear, to a posterior side of the patients ear by passing through an area superior to the patient's ear.

### 1 CROSS REFERENCE TO RELATED APPLICATIONS

This application claims the benefit of Australian Provisional Patent Application No. 2023900972, filed 4 Apr. 2023, and Australian Provisional Patent No. 2023901670, filed 29 May 2023, the contents of which are herein incorporated by reference in their entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and Its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem, e.g. see U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload, e.g. see U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

Certain masks may cause some patients a feeling of claustrophobia, unease and/or may feel overly obtrusive.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

Consequently, some masks suffer from being obtrusive, aesthetically undesirable, costly, poorly fitting, difficult to use, and/or uncomfortable especially when worn for long or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

A range of patient interface seal-forming structure technologies are disclosed in the following patent applications: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. has manufactured the following products that incorporate nasal pillows: SWIFTTM nasal pillows mask, SWIFTTM II nasal pillows mask, SWIFTTM LT nasal pillows mask, SWIFTTM FX nasal pillows mask and MIRAGE LIBERTYTM full-face mask. The following patent applications describe examples of nasal pillows masks: International Patent Application WO 2004/073778 (describing amongst other things aspects of the SWIFTTM nasal pillows mask), US Patent Application 2009/0044808 (describing amongst other things aspects of the SWIFTTM LT nasal pillows mask); International Patent Applications WO 2005/063328 and WO 2006/130903 (describing amongst other things aspects of the MIRAGE LIBERTYTM full-face mask); International Patent Application WO 2009/052560 (describing amongst other things aspects of the SWIFTTM FX nasal pillows mask).

### 2.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives, e.g. see US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

### 2.2.3.1.4 Pressurised Air Conduit Used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a positioning and stabilising structure configured for use in supporting a patient interface in sealing engagement with the face of a patient during delivery of a flow of breathable gas to the nose and mouth of the patient during a respiratory therapy treatment.

Another aspect of one form of the present technology is a positioning and stabilising structure configured for use in supporting a patient interface in sealing engagement with the face of a patient during delivery of a flow of breathable gas to the nose and mouth of the patient during a respiratory therapy treatment, which includes a rigidiser that includes a first end which overlays or sits inferior to the cheekbones of the patient in use, and a second end which is located posterior to the ear of the patient in use.

Another aspect of one form of the present technology is a positioning and stabilising structure configured for use in supporting a patient interface in sealing engagement with the face of a patient during delivery of a flow of breathable gas to the nose and mouth of the patient during a respiratory therapy treatment, the positioning and stabilising structure comprising: a rear strap configured to, in use overlay the occipital bone, and/or trapezius muscles at the rear of the patients head; a top strap, configured to, in use extend across the head of the patient in use, at least one side strap positioned on each side of the positioning and stabilising structure and being configured to connect to the patient interface in use, wherein the at least one side straps are configured to extend from the rear strap and from the top strap along a respective side of the patient's face, superior to the ears of the patient, wherein the at least one side strap comprises a rigidiser, and wherein the rigidiser has a first end which overlays or sits inferior to the cheekbones of the patient in use, and a second end which is located posterior to the ear of the patient in use.

Another aspect of one form of the present technology is a rigidiser for use in a positioning and stabilising structure.

Another aspect of one form of the present technology is a rigidiser for use in a positioning and stabilising structure that includes a rear strap configured to, in use overlay the occipital bone, and/or trapezius muscles at the rear of the patient's head; a top strap, configured to, in use extend across the head of the patient, at least one side strap positioned on each side of the positioning and stabilising structure and being configured to connect to a patient interface in use, wherein the at least one side straps are configured to extend from the rear strap and from the top strap along a respective side of the patient's face, superior to the ears of the patient, the rigidiser comprising a first end which is configured to overlay or sit inferior to the cheekbones of the patient in use, and a second end which is located posterior to the ear of the patient in use.

Another aspect of one form of the present technology is a headgear assembly configured to position and support a patient interface in sealing engagement with the face of a patient during delivery of a flow of breathable gas to the nose and mouth of the patient during a respiratory therapy treatment.

Another aspect of one form of the present technology is a two-point headgear arrangement.

Another aspect of one form of the present technology is a four-point headgear arrangement.

Another aspect of one form of the present technology is a headgear assembly configured to position and support a patient interface in sealing engagement with the face of a patient during delivery of a flow of breathable gas to the nose and mouth of the patient during a respiratory therapy treatment, the headgear assembly comprising: a rear strap configured to, in use overlay the occipital bone, and/or trapezius muscles at the rear of the patients head; a top strap, configured to, in use extend across the patients head, at least one side strap positioned on each side of the headgear assembly and being configured to connect to the patient interface in use, wherein the at least one side straps are configured to extend from the rear strap, and top strap along a respective side of the patients face, such that no straps are positioned inferior to the ears of the patient in use.

Another aspect of one form of the present technology is a positioning and stabilising structure configured for use in supporting a patient interface in sealing engagement with the face of a patient during delivery of a flow of breathable gas to the nose and mouth of the patient during a respiratory therapy treatment, the positioning and stabilising structure comprising: at least one rear strap configured to, in use, overlay the occipital bone and/or trapezius muscles at the rear of the patient's head; a top strap, configured to, in use extend across the head of the user; at least one side strap positioned on each side of the positioning and stabilising structure and being configured to connect to the patient interface in use, wherein the at least one side strap is configured to extend from the rear strap and top strap, at a point which is superior to the ears of the patient, through to the patient interface, along a respective side of the patient's face, wherein the at least one side strap comprises a rigidiser, and wherein the rigidiser extends from a point which is superior to the patient's ear to a point which is inferior and anterior of the patient's ear in use.

Another aspect of one form of the present technology is a respiratory therapy system, which comprises, a positioning and stabilising structure; a patient interface configured to provide a seal with the airways of a patient for delivery of a flow of breathable gas at a positive pressure; a pressure generator configured to generate the flow of breathable gas at a positive pressure; and an air circuit configured to fluidly couple the pressure generator to the patient interface; wherein the positioning and stabilising structure comprises: a rear strap configured to, in use, overlay the occipital bone and/or trapezius muscles at the rear of the patient's head; a top strap, configured to, in use extend across the head of the user; at least one side strap positioned on each side of the positioning and stabilising structure and being configured to connect to the patient interface in use, wherein the at least one side strap is configured to extend from the rear strap and top strap, at a point which is superior to the ears of the patient, through to the patient interface, along a respective side of the patient's face, wherein the at least one side strap each comprises a rigidiser, and wherein the rigidiser has a first end which overlays or sits inferior to the cheekbones of the patient in use, and a second end which is located posterior to the ear of the patient in use.

Another aspect of one form of the technology, is a rigidised strap for a positioning and stabilising structure.

In some aspects, the rigidised strap may comprise a patient contacting layer, a non-patient contacting layer, and a rigidiser configured to in use increase the rigidity of the strap.

In some aspects, the rigidised strap may comprise a first limb and a second limb, the first and second limb being offset laterally from a longitudinal axis of the strap. In some examples the first limb and second limb may be spaced apart to provide a gap between the first limb and the second limb.

In some aspects, a first end of the first limb may be connected to a first end of the second limb, and a second end of the first limb, may be connected to a second end of the second limb. Alternatively, a first end of the first limb may be connected to a first end of the second limb, and a second end of the first limb, may be separated from the second end of the second limb.

Another aspect of the technology is a headgear assembly configured to position and stabilise a patient interface in sealing engagement with the face of a patient during delivery of a flow of breathable gas to the nose and mouth of the patient during a respiratory therapy treatment.

In some aspects of the technology, the headgear may comprise: a rear strap configured to, in use overlay the occipital bone, and/or trapezius muscles at the rear of the patients head; a top strap, configured to, in use overlay the parietal bone on the top of the patients head, and at least one rigidised strap.

In some aspects of the technology, each of the rigidised straps may be configured to extend from the rear strap and top strap, at a point which is superior to the ears of the patient, through to the patient interface, along a respective side of the patient's face.

In some aspects of the technology, each of the rigidised straps may comprise a first end which overlays or sits inferior to the cheekbones of the patient in use, and a second end which is located posterior to the ear of the patient in use.

Another aspect of one form of the technology, is a respiratory therapy system, which comprises: a positioning and stabilising structure; a patient interface configured to provide a seal with the airways of a patient for delivery of a flow of breathable gas at a positive pressure; a pressure generator configured to generate the flow of breathable gas at a positive pressure; and an air circuit configured to fluidly couple the pressure generator to the patient interface.

In some aspects of the technology, the positioning and stabilising structure may comprise: a rear strap configured to, in use, overlay the occipital bone and/or trapezius muscles at the rear of the patient's head; a top strap, configured to, in use extend across the head of the user; and at least one rigidised strap as described herein.

In some aspects of the technology, the patient interface may be configured to deliver the flow of breathable gas to the nasal and oral airways of the patient in use.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure, and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

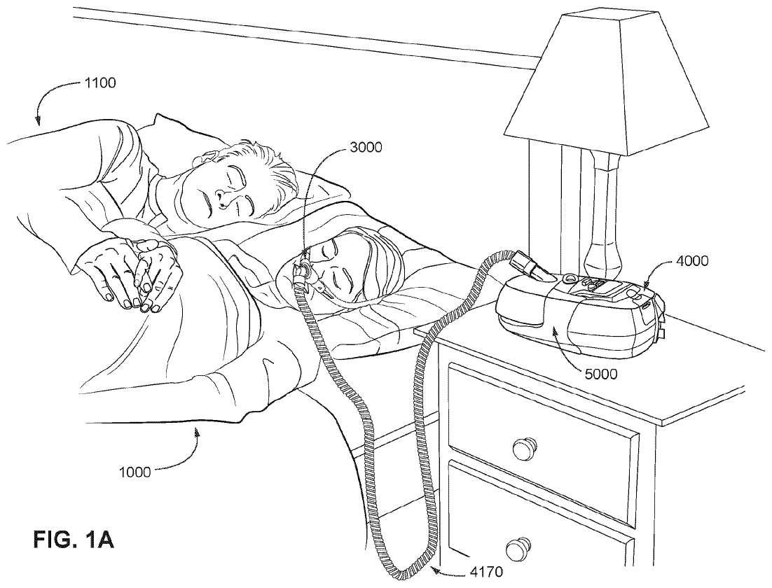

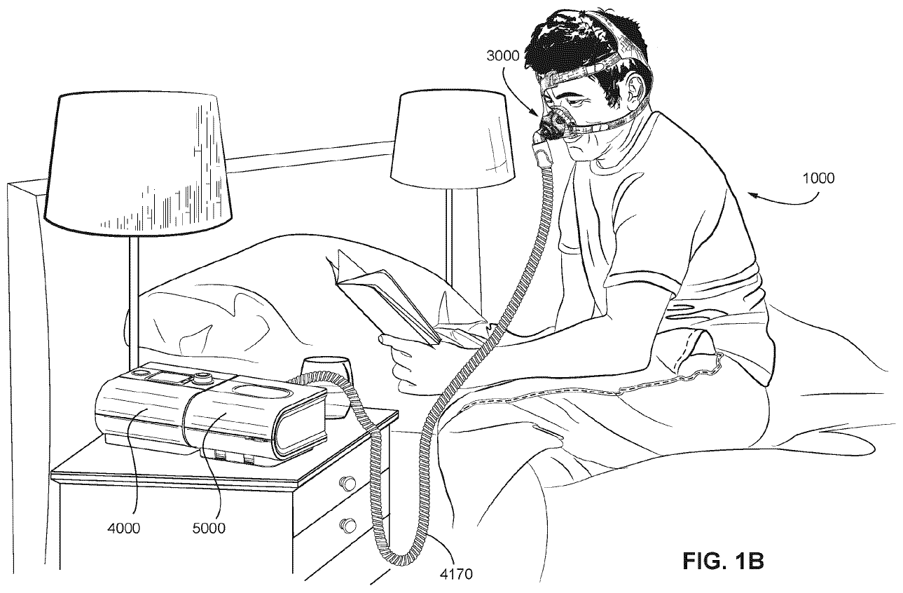

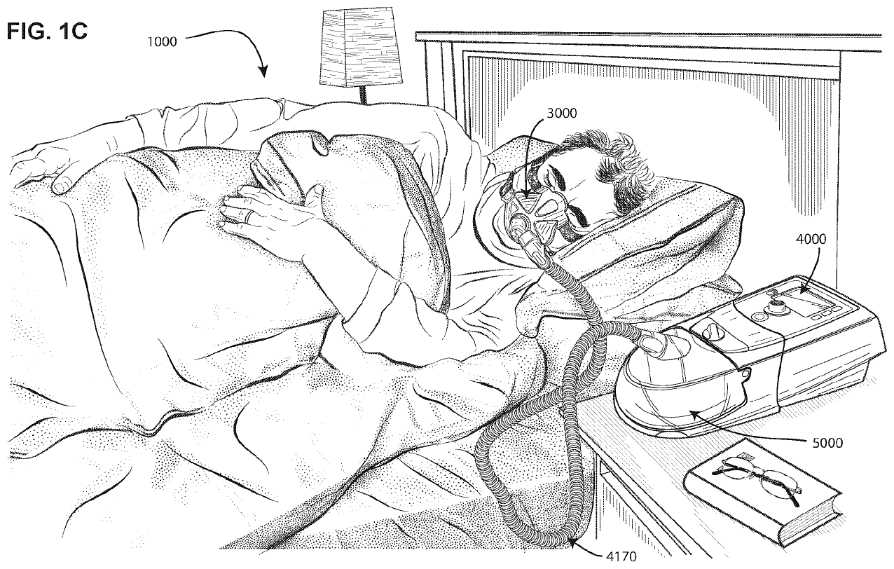

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

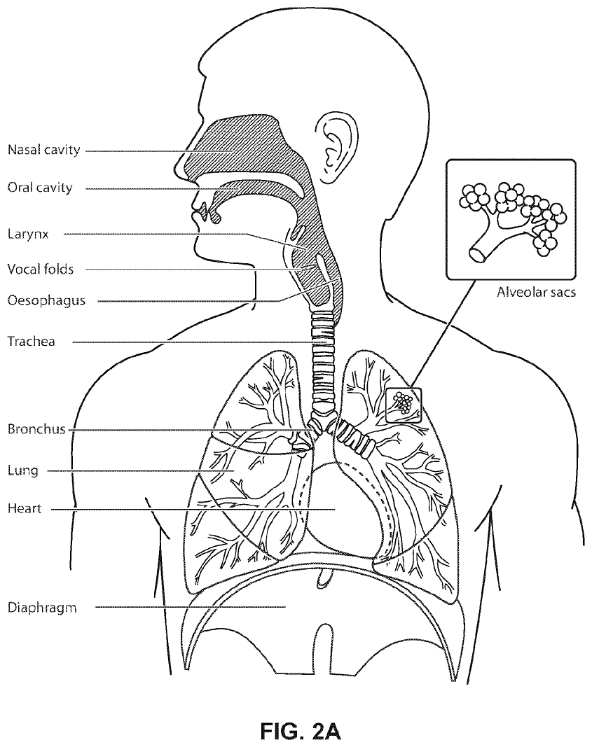

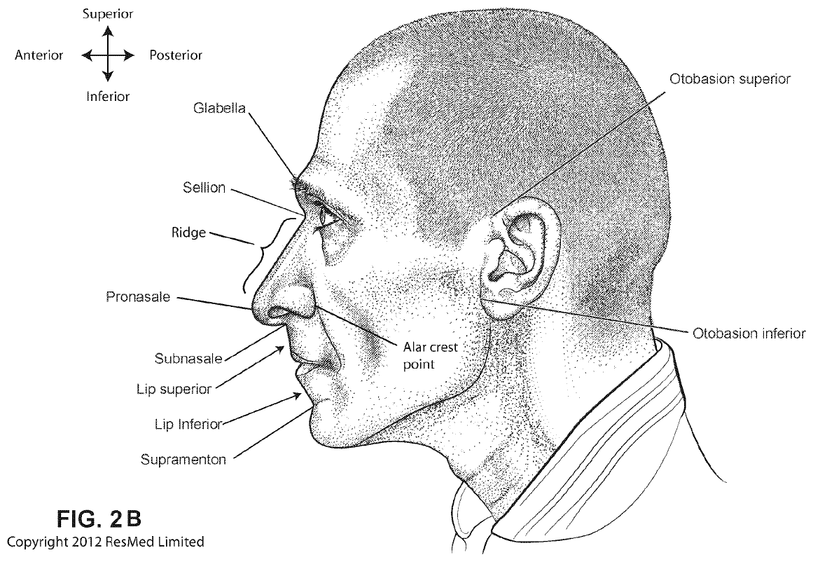

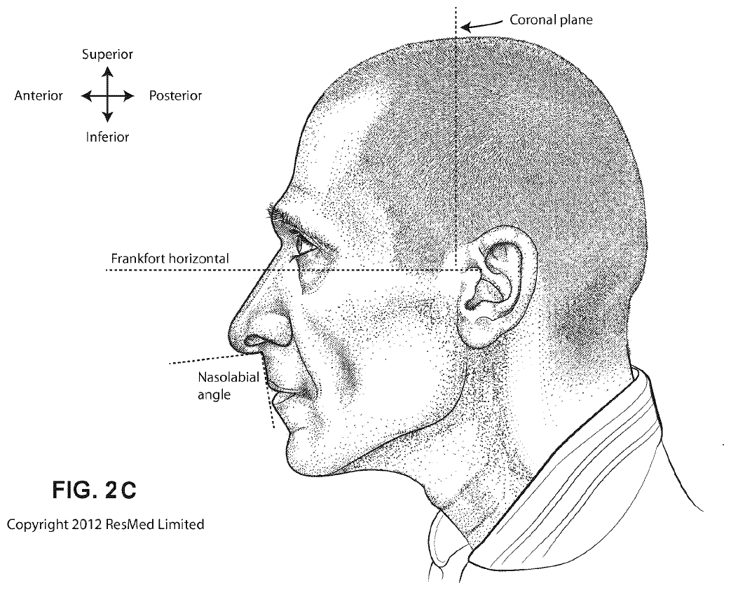

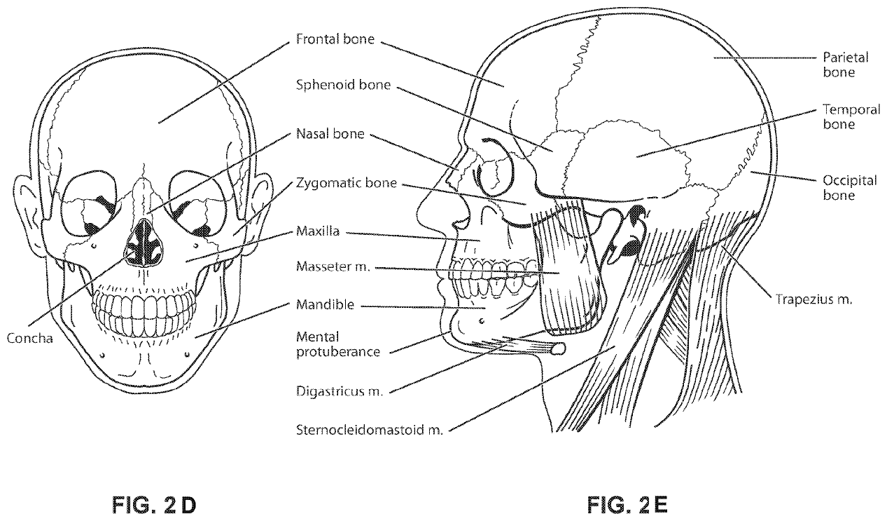

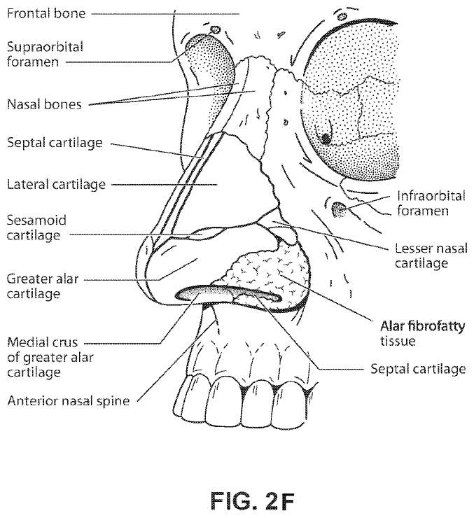

FIG. 2A shows an overview of a human respiratory system including the nasal and oral cavities, the larynx, vocal folds, oesophagus, trachea, bronchus, lung, alveolar sacs, heart and diaphragm.

FIG. 2B is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2C is a further side view of a head. The approximate locations of the Frankfort horizontal and nasolabial angle are indicated. The coronal plane is also indicated.

FIG. 2D shows a front view of the bones of a skull including the frontal, nasal and zygomatic bones. Nasal concha are indicated, as are the maxilla, and mandible.

FIG. 2E shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideo trapezius.

FIG. 2F shows an anterolateral view of a nose.

### 4.3 Patient Interface

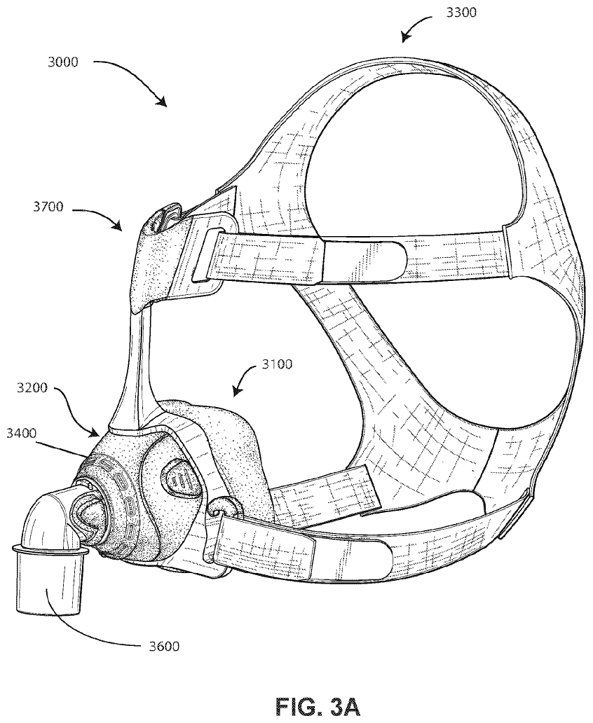

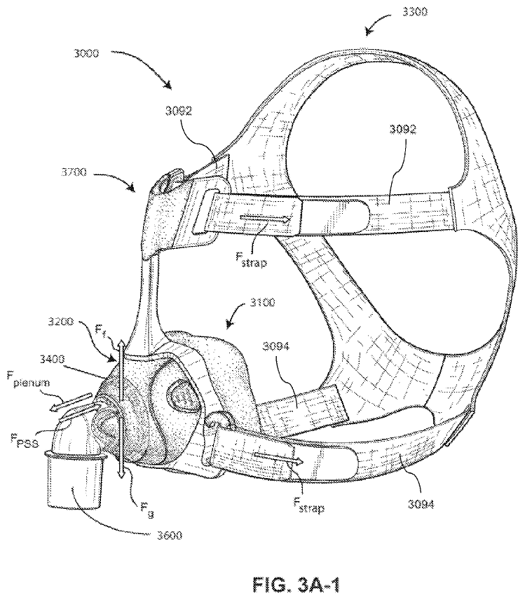

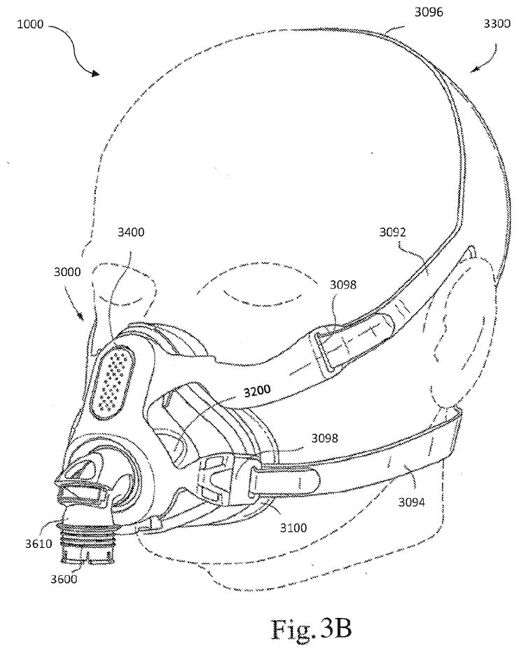

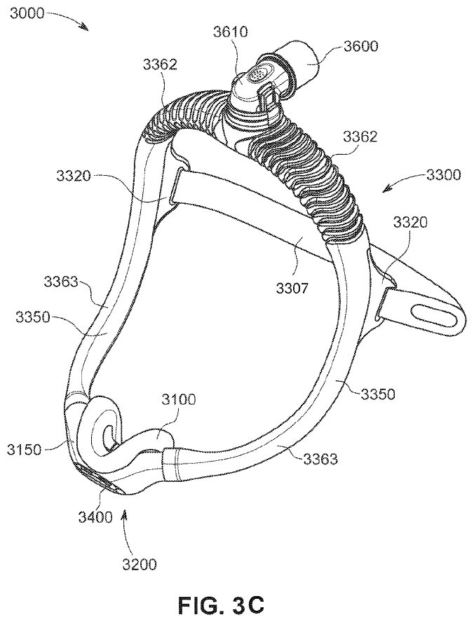

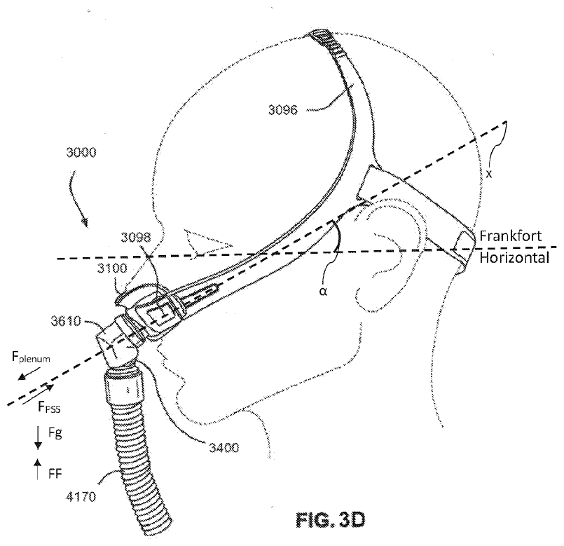

FIG. 3A shows a patient interface in the form of a nasal mask in accordance with one form of the present technology.

FIG. 3A-1 shows forces acting on the patient interface of FIG. 3A, while in use.

FIG. 3B shows a patient interface in the form of a full-face mask in accordance with one form of the present technology.

FIG. 3C shows a patient interface having conduit headgear, in accordance with one form of the present technology.

FIG. 3D shows a side view of headgear supporting a nasal patient interface, in accordance with one form of the present technology.

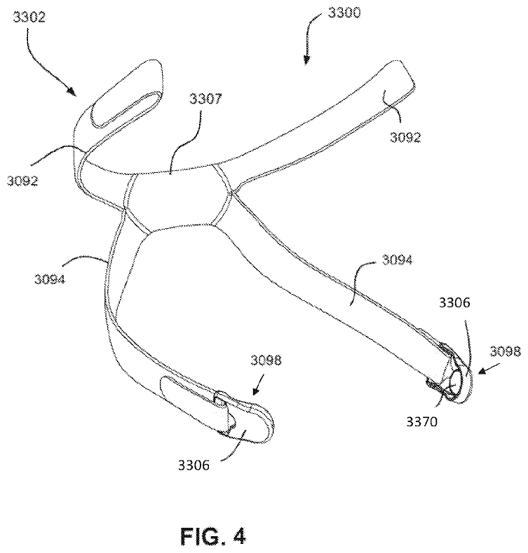

FIG. 4 shows a perspective view of a positioning and stabilising structure comprising headgear straps for supporting a patient interface in accordance with one form of the present technology.

### 4.4 Over-Ear Headgear

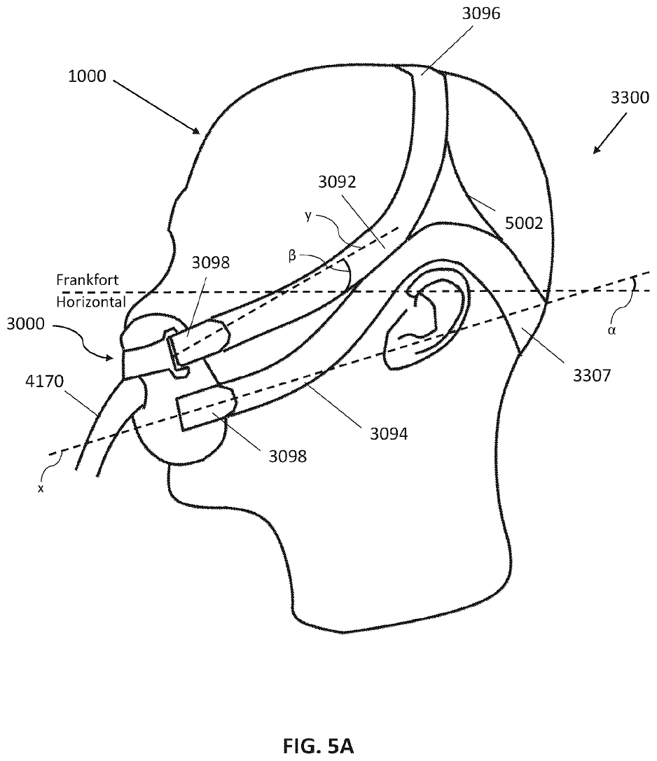

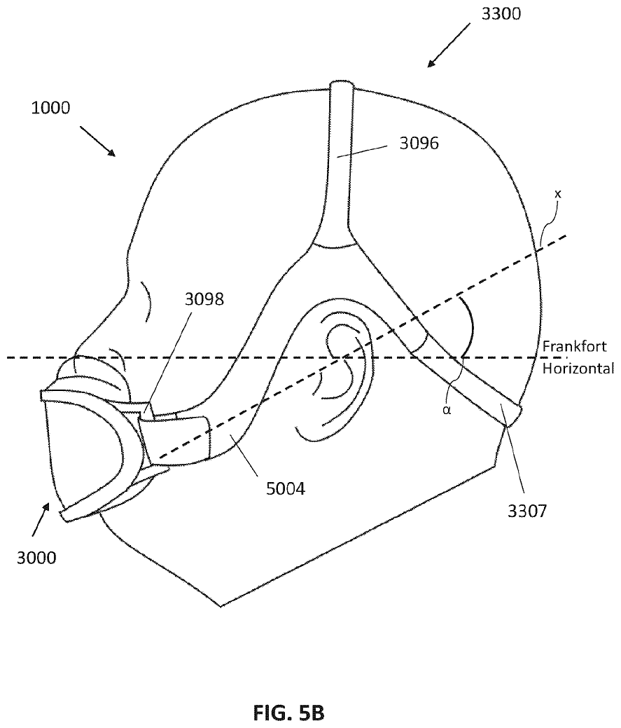

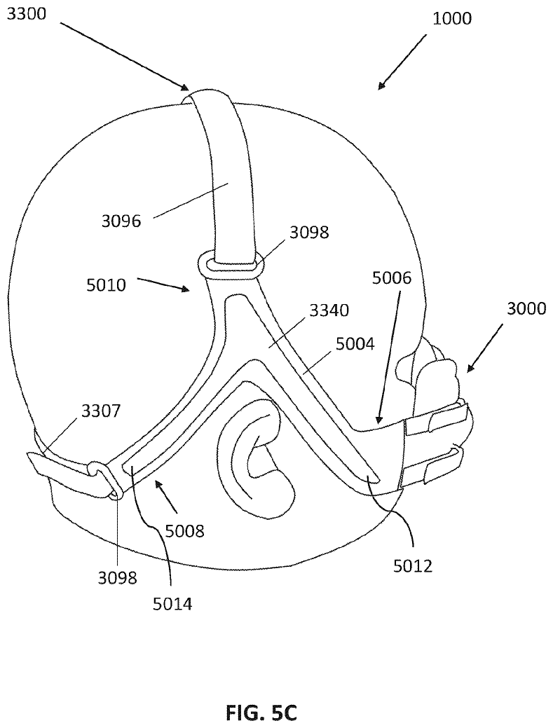

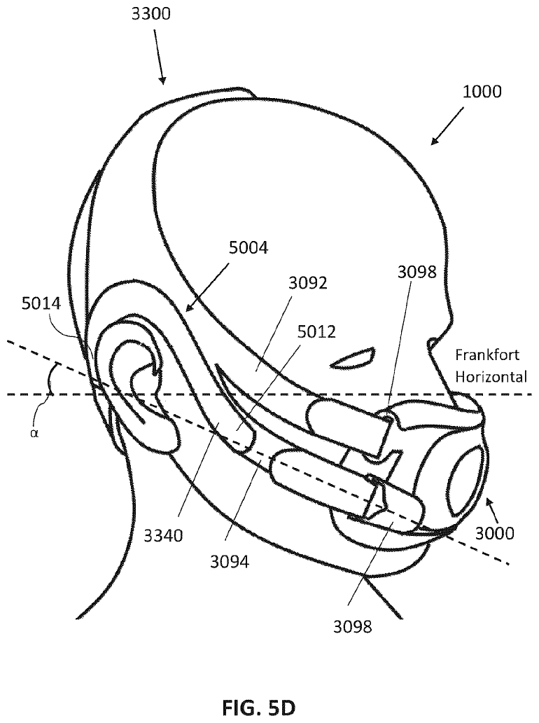

FIG. 5A shows a side view of a four-point positioning and stabilising structure comprising straps which a positioned superior to the ear of the patient in use.

FIG. 5B shows a two-point positioning and stabilising structure in accordance with one form of the present technology.

FIG. 5C shows a side view of a positioning and stabilising structure comprising rigidised straps which a positioned superior to the ear of the patient in use.

FIG. 5D shows a perspective view of a further positioning and stabilising structure comprising a rigidiser which extends from a region anterior to the ear of the patient, to a region posterior of the ear of the patient, by following a path adjacent to the otobasion superior region of the patient.

### 4.5 Rigidiser Configuration

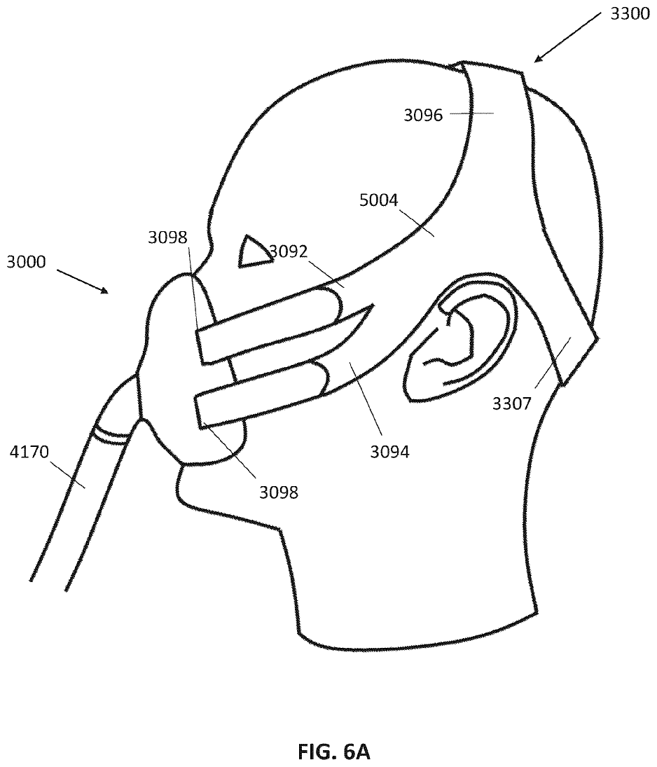

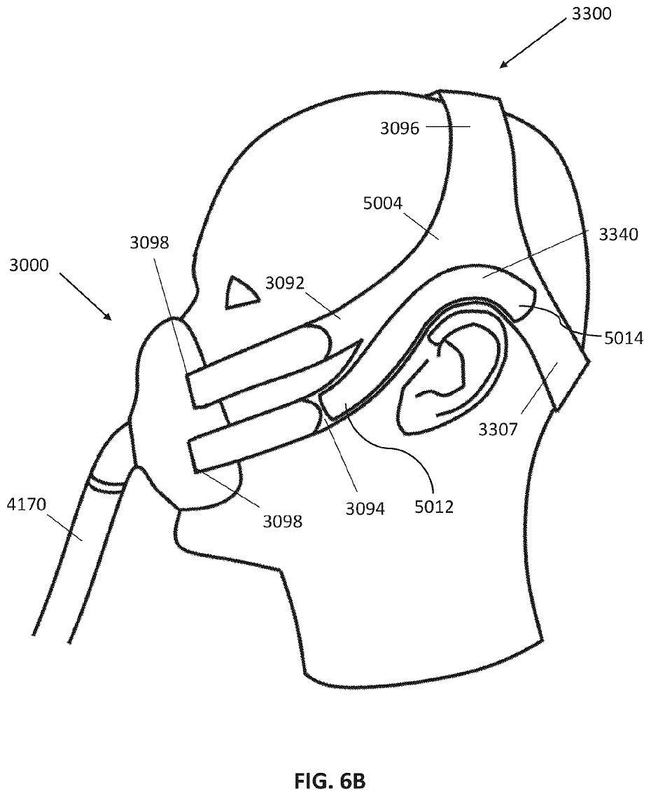

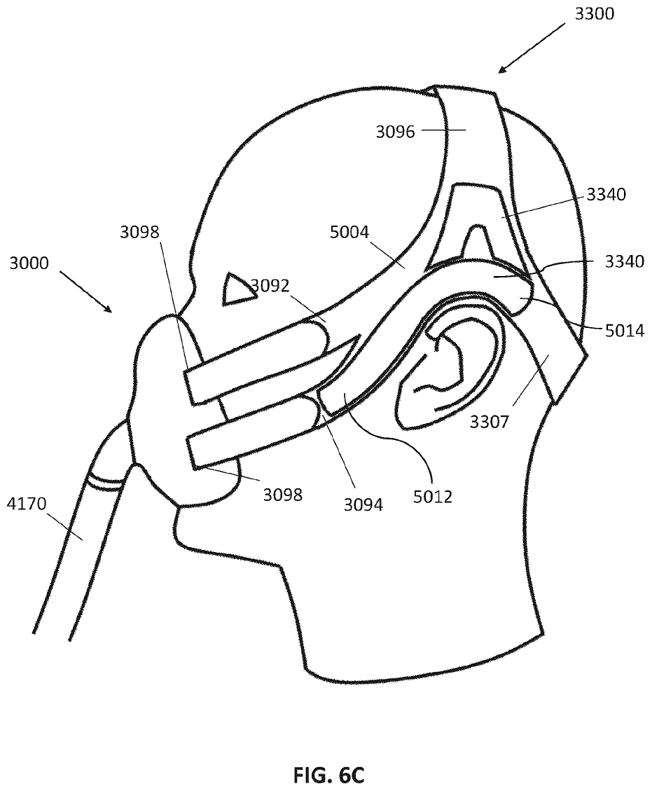

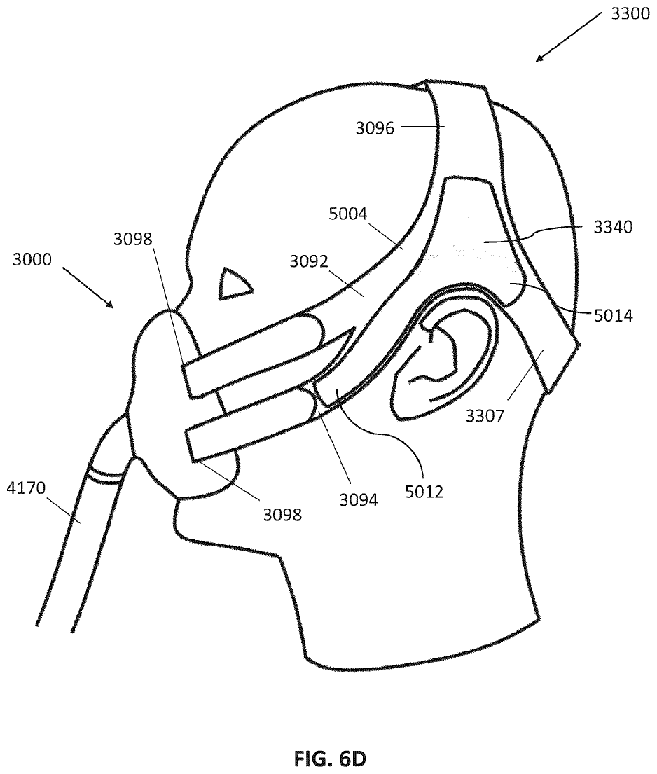

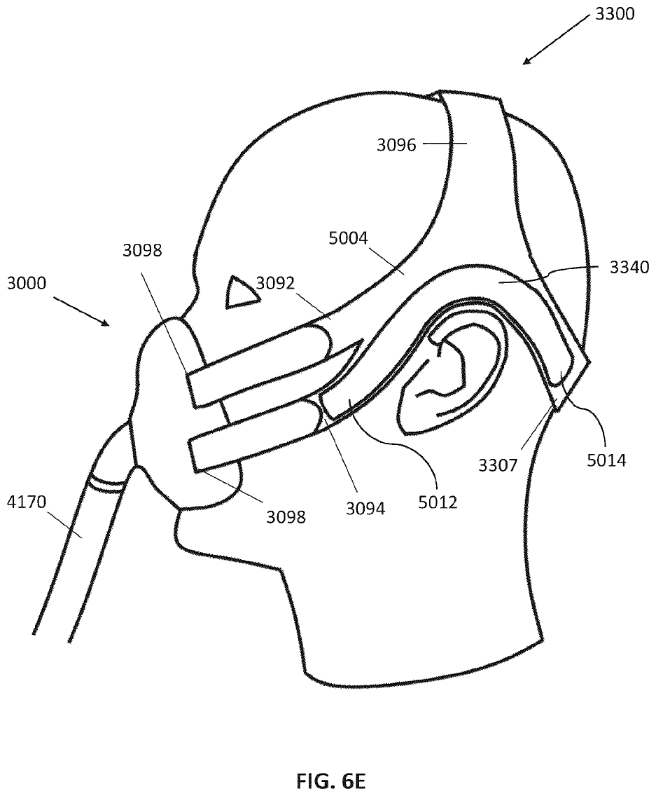

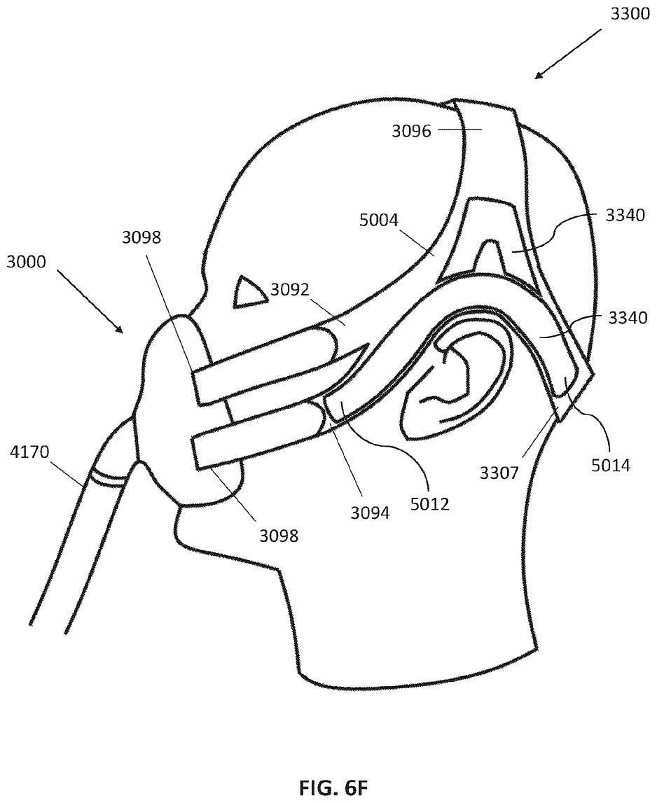

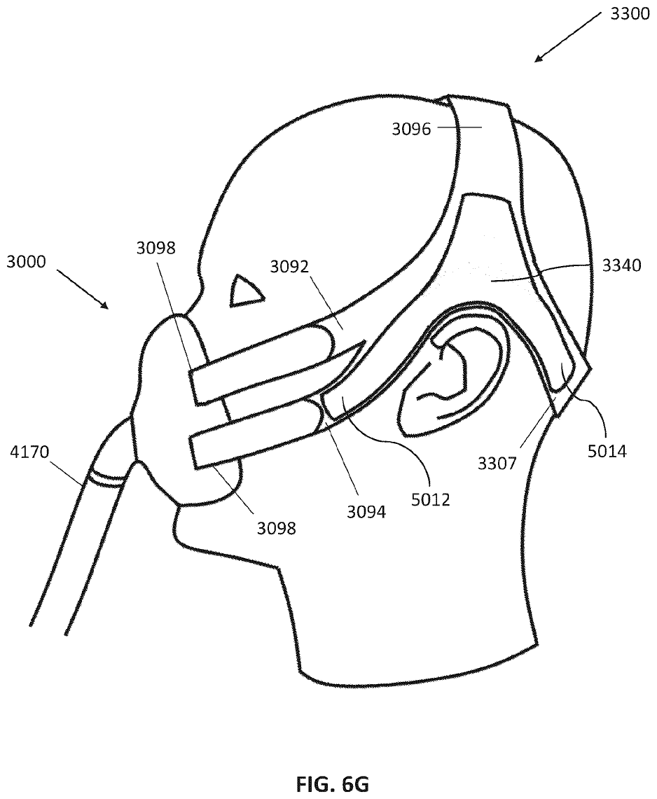

FIG. 6A shows a side view of a positioning and stabilising structure in accordance with one form of the technology.

FIG. 6B shows one form of a rigidiser which may be used with the positioning and stabilising structure of FIG. 6A.

FIG. 6C shows another form of a rigidiser which may be used with the positioning and stabilising structure of FIG. 6A.

FIG. 6D shows another form of a rigidiser which may be used with the positioning and stabilising structure of FIG. 6A.

FIG. 6E shows another form of a rigidiser which may be used with the positioning and stabilising structure of FIG. 6A.

FIG. 6F shows another form of a rigidiser which may be used with the positioning and stabilising structure of FIG. 6A.

FIG. 6G shows another form of a rigidiser which may be used with the positioning and stabilising structure of FIG. 6A.

### 4.6 Rigidiser Construction

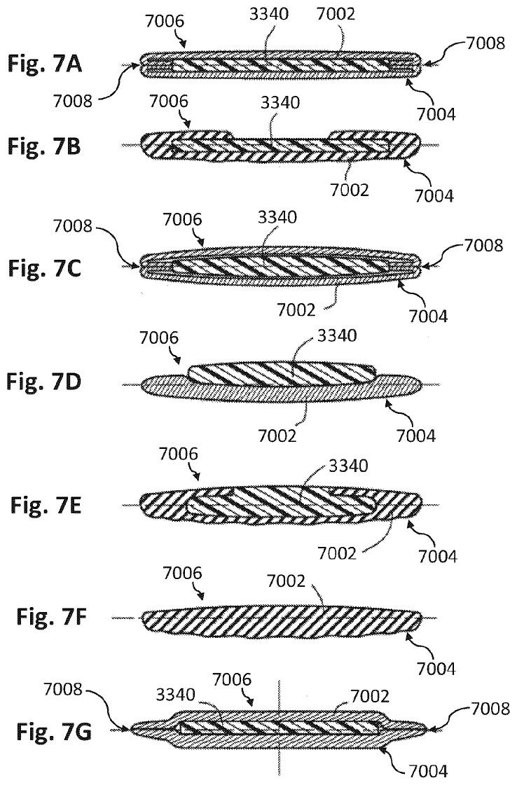

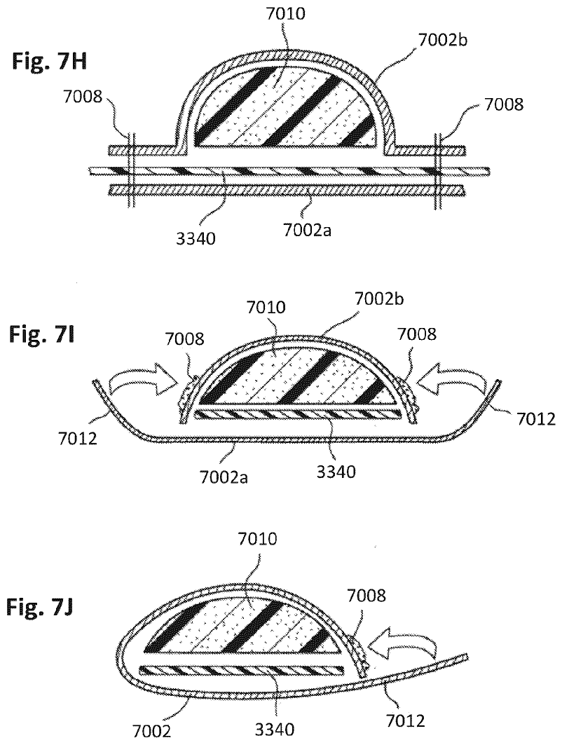

FIG. 7A shows a cross sectional view of a strap comprising a rigidiser in accordance with one form of the technology.

FIG. 7B shows a cross sectional view of a strap comprising a rigidiser in accordance with another form of the technology.

FIG. 7C shows a cross sectional view of a strap comprising a rigidiser in accordance with another form of the technology.

FIG. 7D shows a cross sectional view of a strap comprising a rigidiser in accordance with another form of the technology.

FIG. 7E shows a cross sectional view of a strap comprising a rigidiser in accordance with another form of the technology.

FIG. 7F shows a cross sectional view of a strap comprising a rigidiser in accordance with another form of the technology.

FIG. 7G shows a cross sectional view of a strap comprising a rigidiser in accordance with another form of the technology.

FIG. 7H shows a cross sectional view of a strap comprising a foam layer in accordance with one form of the technology.

FIG. 7I shows a cross sectional view of a strap comprising a foam layer in accordance with another form of the technology.

FIG. 7J shows a cross sectional view of a strap comprising a foam layer in accordance with another form of the technology.

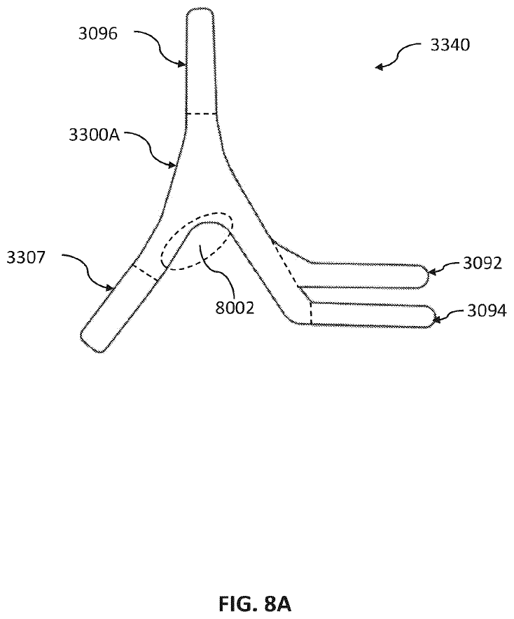

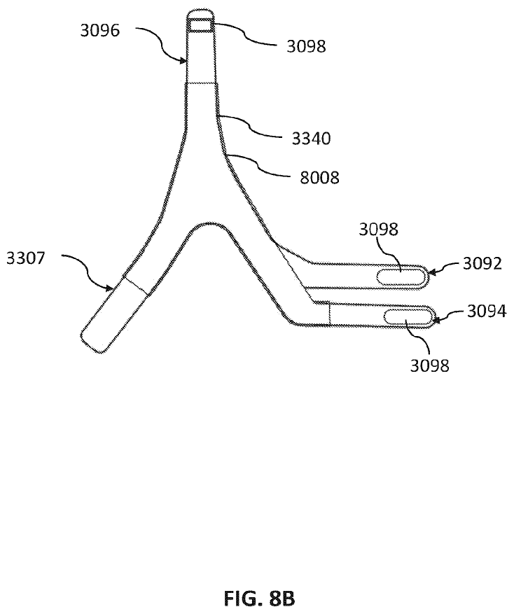

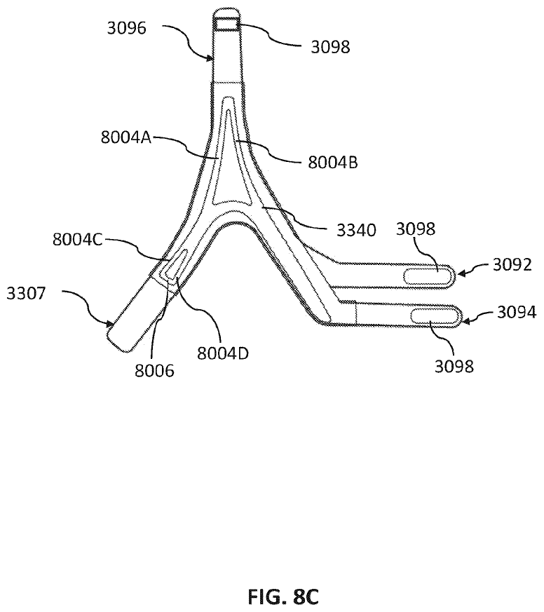

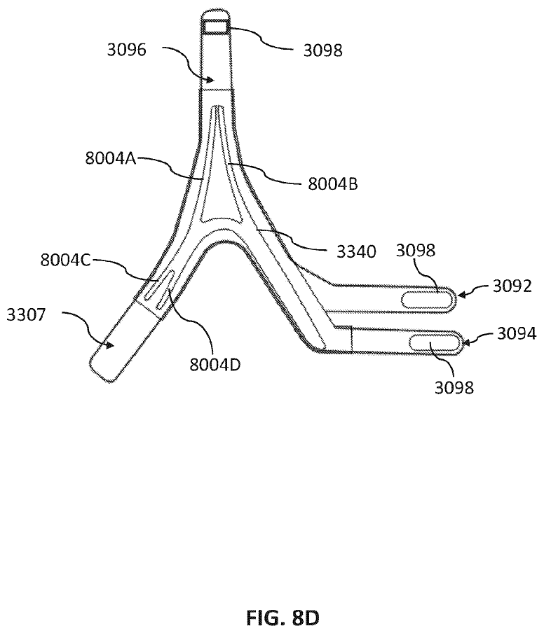

FIG. 8A shows an example of a rigidised headgear strap comprising a plurality of regions in accordance with one example of the technology.

FIG. 8B shows a further example of a rigidised headgear strap in accordance with one form of the technology.

FIG. 8C shows an exemplary rigidiser for a rigidised headgear strap in accordance with one form of the technology.

FIG. 8D shows an exemplary rigidiser for a rigidised headgear strap in accordance with another form of the technology.

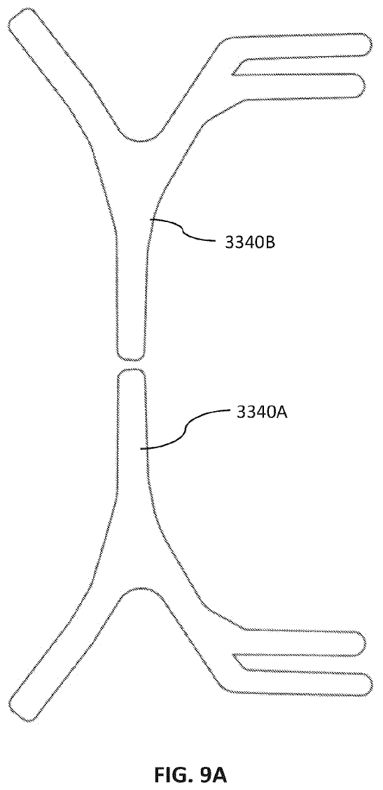

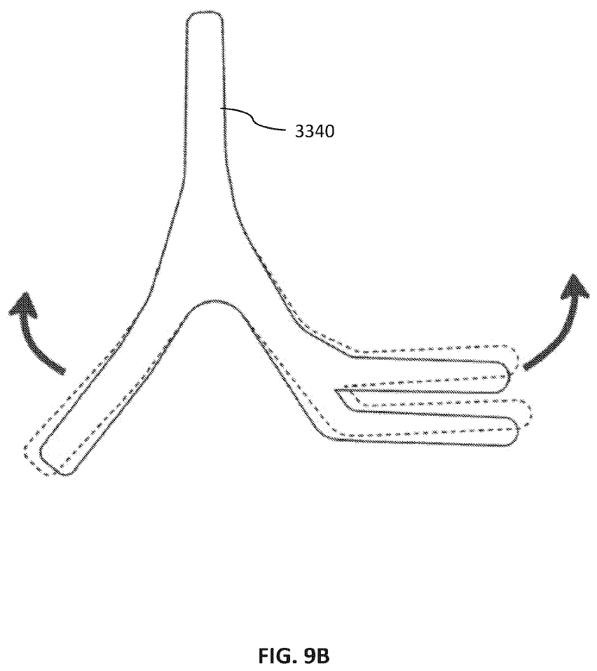

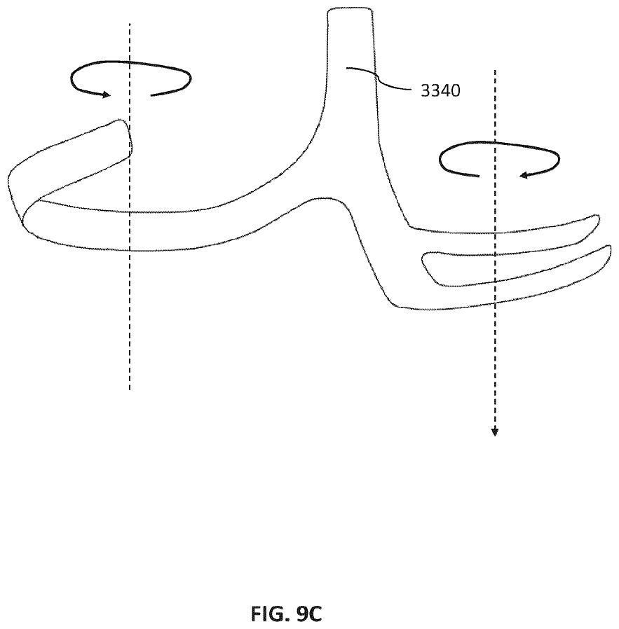

FIG. 9A shows an example of a rigidiser template in accordance with one form of the technology.

FIG. 9B shows an example of in-plane bending in accordance with one form of the technology.

FIG. 9C shows an example of out-of-plane bending in accordance with another form of the technology.

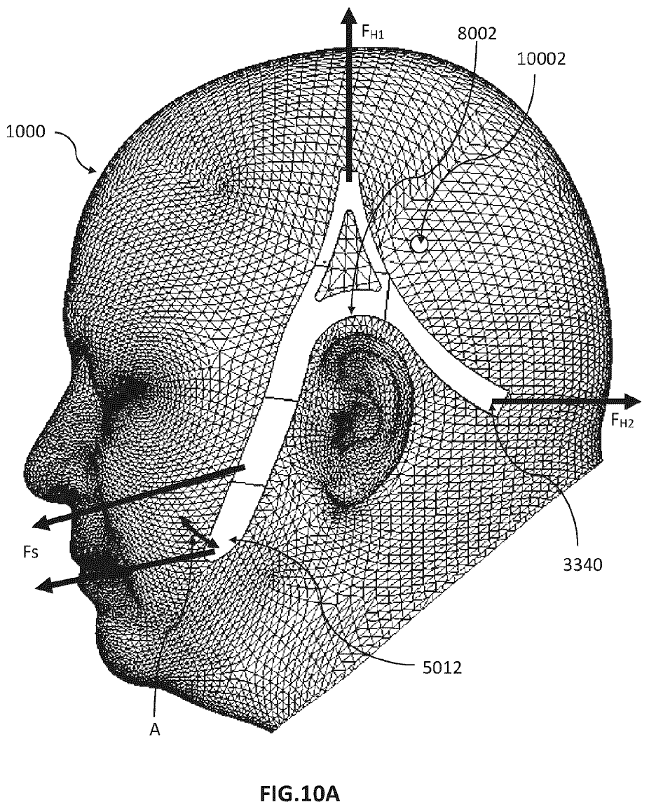

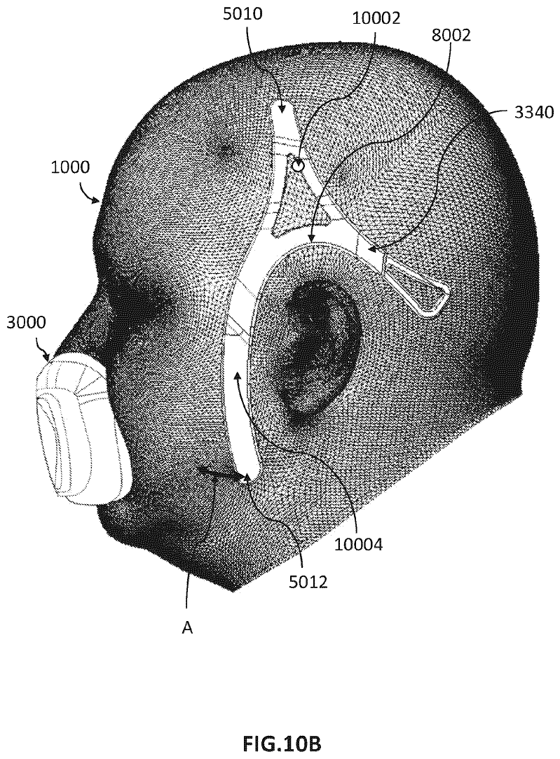

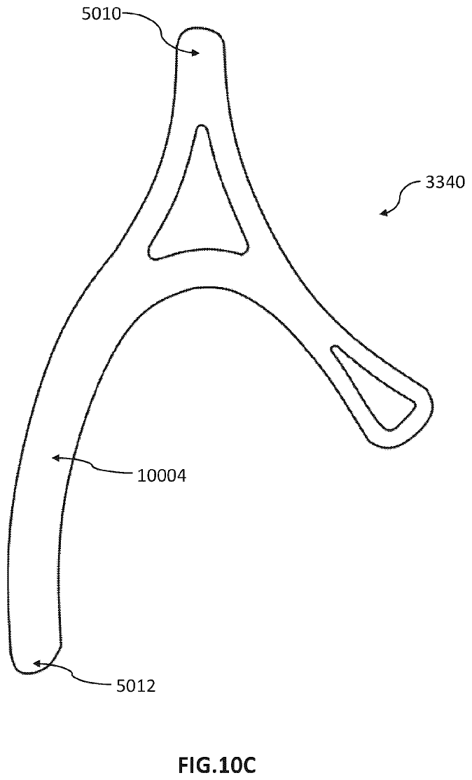

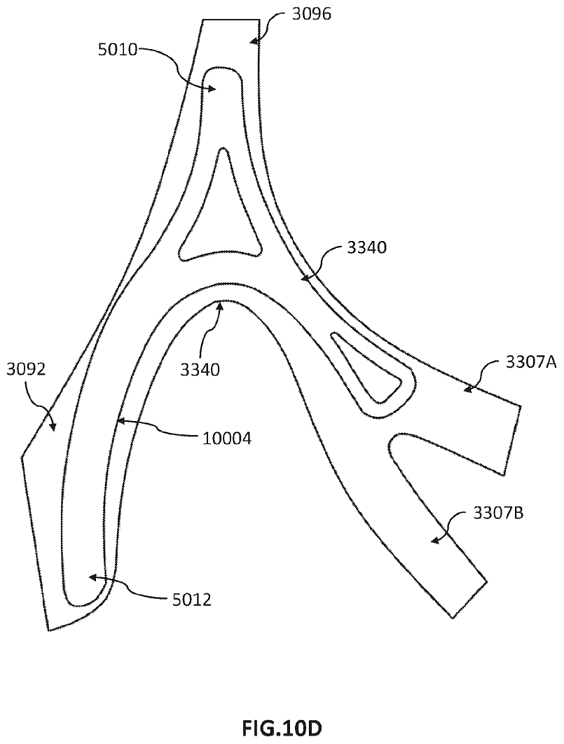

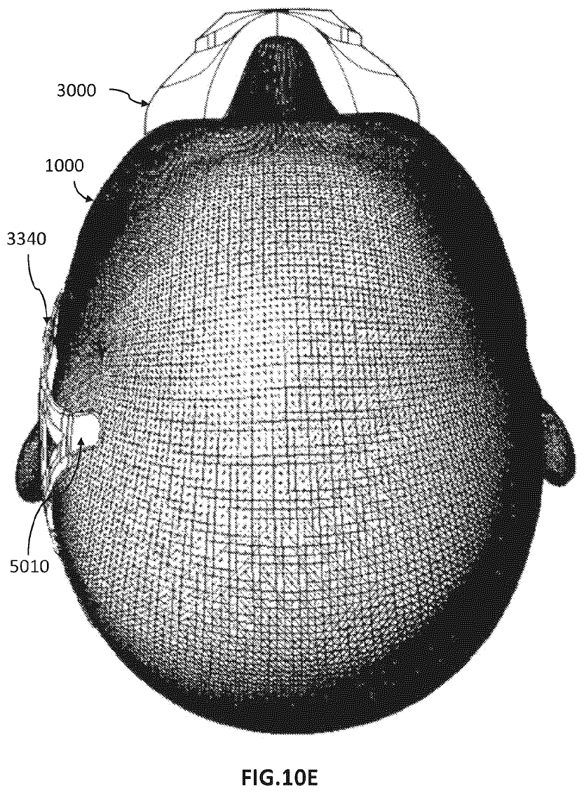

FIG. 10A shows a side view of a rigidiser against a patient's head in accordance with one form of the technology.

FIG. 10B shows a side view of another rigidiser against a patient's head in accordance with another form of the technology.

FIG. 10C shows a side view of the rigidiser of FIG. 10B.

FIG. 10D shows a side view of the rigidiser of FIG. 10B, overlaid on, or positioned within headgear.

FIG. 10E shows a top view of a patient's head with the rigidiser of FIG. 10B positioned against one side.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares. While in other examples, the supply of air is provided to the oral and nasal passages of the patient.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

Non-invasive patient interfaces 3000, such as those shown in FIG. 3A and FIG. 3B, in accordance with one aspect of the present technology comprises the following functional aspects: a seal-forming structure 3100, a plenum chamber 3200, a vent 3400, one form of connection port 3600 for connection to air circuit 4170, and an optional forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

In use the patient interface is supported on the patient's head via a positioning and a positioning and stabilising structure 3300, or headgear.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH2O with respect to ambient.

### 5.3.1 Seal-Forming Structure

In one form, such as the example illustrated in FIG. 3B, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 3B is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form illustrated in FIG. 5A the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above-described examples of nose-only and nose and mouth masks.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

### 5.3.3 Positioning and Stabilising Structure

The seal-forming structure 3100 of the patient interface 3000 of the present technology may be held in sealing position in use by a positioning and stabilising structure 3300. The positioning and stabilising structure 3300 may comprise and function as “headgear” since it engages the patient's head in order to hold the patient interface 3000 in a sealing position. Examples of a positioning and stabilising structure may be shown in FIGS. 3A and 4.

In one form the positioning and stabilising structure 3300 provides a retention force at least sufficient to overcome the effect of the positive pressure in the plenum chamber 3200 to lift off the face (i.e., Fₚₗₑₙᵤₘ).

In one form the positioning and stabilising structure 3300 provides a retention force to overcome the effect of the gravitational force (i.e., F_{g}) on the patient interface 3000.

With reference to FIG. 3A-1, and FIG. 3D, the positioning and stabilising structure 3300 provides a force F_{PSS} that assists in maintaining the plenum chamber 3200 in the sealing position on the patient's face. The positioning and stabilising force F_{PSS} may be the resultant force from the various forces of the different elements of the positioning and stabilising structure 3300. For example, the upper headgear straps 3092, and lower headgear straps 3094 may individually or together provide a strap force Fₛₜᵣₐₚ in order to hold the seal-forming structure 3100 against the patient's face. The force Fₛₜᵣₐₚ may also be directed at least partially in the superior direction in order to overcome the gravitational force F_{g}. The gravitational force F_{g} may be specifically shown for the seal-forming structure 3100 and the plenum chamber 3200, but gravity would act on the entirely of the patient interface 3000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the seal-forming structure 3100 and the plenum chamber 3200 in the inferior direction (as viewed in FIG. 3A-1), the frictional force F_{f} would act in the superior direction (e.g., against a patient's face). For example, the patient may experience the frictional force Fr against his lip superior (and/or other surfaces of the patient's face in contact with the seal-forming structure 3100) in order to oppose the motion in the inferior direction (which may help to stabilising the cushion in place). Although the frictional force F_{f} is shown specifically opposing the gravitational force F_{g} of the seal-forming structure 3100 and the plenum chamber 3200, components of an overall frictional force (not shown) would also oppose the gravitational force F_{g} associated with the positioning and stabilising structure 3300 and any other portions of the patient interface 3000. A force of friction can act along any place where the patient interface 3000 contacts the patient's skin (or hair). The frictional force F_{f} extends in the opposite direction of the gravitational force F_{g} and along the patient's skin (or hair). In some forms the gravitational force F_{g} may also be countered by vertical components of the reaction force from the patient's face acting on the seal-forming structure 3100, for example at the nose ridge and chin regions of the patient's face, for example.

In some forms, the sum of the various forces may equal zero so that the patient interface 3000 is at equilibrium (e.g., not moving along the patient's face while in use). Specifically, the gravitational force F_{g} and the blowout force Fₚₗₑₙᵤₘ tend to move the seal-forming structure 3100 away from the desired sealing position. The positioning and stabilising force F_{PSS} is applied in order to counteract the gravitational force F_{g} and the blowout force Fₚₗₑₙᵤₘ (as well as any frictional forces F_{f}) and keep the seal-forming structure 3100 properly situated. Although the positioning and stabilising force F_{PSS} may exceed the sum of the gravitational force F_{g} and the blowout force Fₚₗₑₙᵤₘ (with any additional positioning and stabilising force F_{PSS} being balanced by reaction force from the patient's head acting on the portions of patient interface 3000) and still maintain the seal-forming structure 3100 in an appropriate sealing position, patient comfort may be sacrificed. Maximum patient comfort may be achieved when the net force on the patient interface 3000 is zero and the positioning and stabilising force F_{PSS} is exactly strong enough to achieve this. In some examples the positioning and stabilising structure 3300 may be adjustable such that when fitted the positioning and stabilising force F_{PSS} is greater than required to exactly balance the gravitational force F_{g} and the blowout force Fₚₗₑₙᵤₘ to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force F_{PSS} necessary to achieve equilibrium.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap 3092, 3094 having a rectangular cross-section, including for example a rounded rectangle which may advantageously lift the edges of the straps outwardly away from the face of the patient, which may help to improve comfort, and/or reduce the occurrence of facial marking on the patient. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example, the strap may be configured in use to be in tension, and to direct a force to draw a seal-forming structure into sealing contact with a portion of a patient's face. In an example the strap may be configured as a tie.

In one form of the present technology, the positioning and stabilising structure comprises a top strap 3096, the top strap 3096 being constructed and arranged so that in use at least a portion of an inferior edge thereof passes superior to an otobasion superior of the patient's head extends from one side of the patients head to the opposing side. For example, the top strap 3096 may extend over the top of the patient's head. In some examples, the top strap 3096 may overlay a portion of a parietal bone of the patient's head in use. In some examples the top strap 3096, may partially overlay the frontal bone of the patient's head in use.

In some examples the top strap 3096 may be superior to the otobasion superior of a patient in use.

In examples, the top may be adjustable, such as having and adjustable length in order to allow the positioning and stabilising structure to be adjusted in a superior-inferior direction relative to the patient's head. Accordingly, the top strap may be used to anchor the positioning and stabilising structure to a top of the patient's head.

In one form of the present technology suitable for a nasal-only mask or for a full-face mask, the positioning and stabilising structure includes a rear strap 3307, the rear strap 3307 being constructed and arranged so that in use, at least a portion of the rear strap 3096 is inferior to the otobasion superior of a patient in use.

In some examples, at least a portion of the rear strap may be configured to pass inferior to an otobasion inferior of the patient's head, or otherwise overlays or lies inferior to the occipital bone of the patient's head.

In one form of the present technology suitable for a nasal-only mask or for a full-face mask, the positioning and stabilising structure may comprise a rear strap 3307 which is continuous with, or otherwise connected to the top strap 3096, such that the combined rear/top strap overlays the parietal and occipital bones of the patient's head in use. The use of a combined or joined rear 3307 and top straps 3096 may reduce a tendency of the rear 3307 and top straps 3096 to move apart from one another.

In examples the rear strap 3307 may comprise an adjustment mechanism which allows the patient to adjust the tension provided by the rear strap on the head. In some examples, it may be beneficial for the rear strap to engage with the rear of the head in a region which is just above or superior to the occipital protuberance.

In certain forms of the present technology, one or more of the straps may be bendable and e.g. non-rigid. An advantage of this aspect is that the non-rigid strap may be more comfortable for a patient to lie upon while the patient is sleeping.

In certain forms of the present technology, any one or more of the straps of the positioning and stabilising structure 3300 may be constructed to be breathable to allow moisture vapour to be transmitted through the strap.

In certain forms of the present technology, a system is provided comprising more than one positioning and stabilising structure 3300, each being configured to provide a retaining force to correspond to a different size and/or shape range. For example, the system may comprise one form of positioning and stabilising structure 3300 suitable for a large sized head, but not a small sized head, and another. suitable for a small sized head, but not a large sized head.

### 5.3.3.1 Conduit Headgear

### 5.3.3.1.1 Conduit Headgear Tubes

In some forms of the present technology, such as the example illustrated in FIG. 3C, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3C, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3C, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 3C, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use.

The tubes 3350 may be formed from a flexible material, such as an elastomer, e.g. silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

In some examples, the one or more tubes 3350 are crush resistant to resist being blocked if crushed during use, for example if squashed between a patient's head and pillow, especially if there is only one tube 3350. The tubes 3350 may be formed with a sufficient structural stiffness to resist crushing or may be as described in U.S. Pat. No. 6,044,844, the contents of which are incorporated herein by reference.

Each tube 3350 may be configured to receive a flow of air from the connection port 3600 on top of the patient's head and to deliver the flow of air to the seal-forming structure 3100 at the entrance of the patient's airways. In the example shown in FIG. 3C, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

Conduits forming part of the positioning and stabilising structure 3300, like headgear straps, may provide a force that contributes to the positioning and stabilising force F_{PSS}. As illustrated in FIG. 3A-1, the positioning and stabilising force F_{PSS} may be the resultant force from the various forces of the different elements of the positioning and stabilising structure 3300. For example, each conduit may provide a force F_{conduit} directed in the posterior and respective lateral direction in order to hold the seal-forming structure 3100 against the patient's face (into the upper lip and sealing under the nose) and oppose the effect of the positive pressure in the plenum chamber 3200 to lift off the face (i.e., Fₚₗₑₙᵤₘ). The force F_{conduit} directed may also be directed at least partially in the superior direction in order to overcome the gravitational force F_{g}.

In some forms, the conduits may provide a force directed into the patient's head when the conduits are filled with pressurized air. The force may assist in gripping the patient's head. The force may be caused by the inflation of the conduits during normal use. In some forms, the force may provide a cushioning effect to the patient's head. The conduits may be designed in order to limit expansion in order to prevent over-gripping the patient's head.

The position of the patient's head may also change the gripping force of the conduits. For example, if the patient is sleeping on his side, the weight of the patient's head may compress one conduit, and the other conduit (e.g., the lateral portion not between the patient's head and a sleeping surface, like a pillow) may additionally expand in order to keep substantially the same flow rate of pressurized air.

The gravitational force F_{g} may be opposed by a frictional force F_{f}, which may act in a direction directly opposite of the gravitational force F_{g}. As gravity pulls the seal-forming structure 3100 and the plenum chamber 3200 in the inferior direction (as viewed in FIG. 3A-1), the frictional force F_{f} would act in the superior direction (e.g., against a patient's face). For example, the patient may experience the frictional force F_{f} against his lip superior (and/or other surfaces of the patient's face in contact with the seal-forming structure 3100) in order to oppose the motion in the inferior direction (which may help to stabilising the cushion in place). Although the frictional force F_{f} is shown specifically opposing the gravitational force F_{g} of the seal-forming structure 3100 and the plenum chamber 3200, components of an overall frictional force (not shown) would also oppose the gravitational force F_{g} associated with the positioning and stabilising structure 3300 and any other portions of the patient interface 3000. A force of friction can act along any place where the patient interface 3000 contacts the patient's skin (or hair). The frictional force F_{f} extends in the opposite direction of the gravitational force F_{g} and along the patient's skin (or hair).

In some forms, the sum of the various forces may equal zero so that the patient interface 3000 is at equilibrium (e.g., not moving along the patient's face while in use). Specifically, the gravitational force F_{g} and the blowout force Fₚₗₑₙᵤₘ tend to move the seal-forming structure 3100 away from the desired sealing position. The positioning and stabilising force F_{PSS} is applied in order to counteract the gravitational force F_{g} and the blowout force Fₚₗₑₙᵤₘ (as well as any frictional forces F_{f}) and keep the seal-forming structure 3100 properly situated. Although the positioning and stabilising force F_{PSS} may exceed the sum of the gravitational force F_{g} and the blowout force Fₚₗₑₙᵤₘ (with any additional positioning and stabilising force F_{PSS} being balanced by reaction force from the patient's head acting on the portions of patient interface 3000) and still maintain the seal-forming structure 3100 in an appropriate sealing position, patient comfort may be sacrificed. Maximum patient comfort may be achieved when the net force on the patient interface 3000 is zero and the positioning and stabilising force F_{PSS} is exactly strong enough to achieve this. In some examples the positioning and stabilising structure 3300 may be adjustable such that when fitted the positioning and stabilising force F_{PSS} is greater than required to exactly balance the gravitational force F_{g} and the blowout force Fₚₗₑₙᵤₘ to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force F_{PSS} necessary to achieve equilibrium.

In examples of the technology herein positioning and stabilising structures are described in the context of straps, and rigidisers. It should be appreciated that any one or more of the examples described the straps may comprise a conduit, and in some examples the rigidisers may be provided by the conduits.

### 5.3.3.2 Headgear Straps

In some forms, the positioning and stabilising structure 3300 may include headgear 3302 comprising at least one strap which may be worn by the patient in order to assist in properly orienting the seal-forming structure 3100 against the patient's face (e.g., in order to limit or prevent leaks).

As described above, some forms of the headgear 3302 may be constructed from a textile material, which may be comfortable against the patient's skin. The textile may be flexible in order to conform to a variety of facial contours, although in some forms the headgear 3302 may include one or more rigidisers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a sealing force for the seal-forming structure 3100.

Two forms of the headgear, two-point headgear and four-point headgear, are discussed in more detail below with respect to FIGS. 3D and 4 respectively.

In some forms, the headgear 3302 may be formed from a continuous piece of material. In other words, the headgear 3302 may not be formed from multiple straps connected (e.g., stitched) together. This may be comfortable for a patient as they will not be in contact with any seams or joints connecting different straps. In other forms, the headgear 3302 may be formed from multiple straps (e.g., two superior side straps, a rear strap, etc.) that are connected together (e.g., with stitching, ultra-sonic welding, etc.).

### 5.3.3.2.1 Four-Point Connection

As shown in FIGS. 3A, 3A-1, 3B and 4, some forms of the positioning and stabilising structures 3300 may be provided as four-point connection headgear. This means that the positioning and stabilising structure 3300 may connect to four separate places on the patient interface 3000, such as on the plenum chamber 3200, on a frame connected to the plenum chamber 3200, and/or on arms connected to the plenum chamber 3200. The positioning and stabilising structures 3300 may include four different side straps providing a tensile force to help maintain the seal-forming structure 3100 in a sealing position, for example an upper/superior strap 3092, and a lower/inferior strap 3094 on each side of the patient's head. In the example of FIGS. 3A and 3A-1, the superior side straps 3092 are connected to a forehead support 3700.

The superior 3092 and inferior 3094 straps each connect to the patient interface 3000 using connection members 3098 which can include any one or more of hook and loop fasteners, clips, buckles, magnetic connectors, or any other connection member familiar to those skilled in the art.

In some forms, the positioning and stabilising structure 3300 may include inferior side straps 3094, which may connect to an inferior portion of the patient interface. The inferior side straps 3094 may extend along the patient's cheek toward a posterior region of the patient's head. For example, the inferior side straps 3094 may overlay the masseter muscle on either side of the patient's face. In examples described herein, the inferior side straps 3094 are configured to extend from the patient interface, over the ear of the patient, to join with or otherwise form the rear strap 3077. For example, inferior side straps 3094 may meet at the posterior of the patient's head where they connect to or otherwise form rear straps 3077 as described herein. and may overlay the occipital bone and/or the trapezius muscle.

The positioning and stabilising structure 3300 may also include side superior side straps 3092, which may overlay the temporal bones, parietal bone, and/or occipital bone.

A rear strap 3307 may extend between the superior side straps 3092 and the inferior side straps 3094. The inferior and superior side straps 3094, 3092 on a given side (e.g., left or right) may also be connected to the rear strap 3307 adjacent to one another. The width of the rear strap 3307, i.e. the distance that the rear strap extends in a superior-inferior direction on the back of the patient's head may therefore be approximately the combined height of the inferior and superior strap 3094, 3092. The rear strap 3307 may overlay the occipital bone and/or the parietal bone in use. This may allow the rear strap 3307 to assist in anchoring the headgear 3302 to the patient's head.

In the illustrated example of FIG. 4, the positioning and stabilising structure 3300 may be formed with a substantially X-shape. The inferior and superior side straps 3094, 3092 may be connected to the rear strap 3307 using stitching, ultrasonic welding, or any similar process. For example, the headgear 3302 may split from the rear strap 3307 into one or more side straps, such as the superior side straps 3092 and inferior side straps 3094. In preferred examples, the superior and inferior side straps each extend above the ears of the patient in use to connect to the patient interface 3000.

In some forms, the superior 3092 and inferior side straps 3094 are configured to connect to the patient interface through one or more connection members 3098. For example, in FIG. 4 the connection members comprise magnetic members 3306. For example, each inferior side straps 3094 may be threaded through the connection members 3098, so that a length of each of the inferior and superior side straps may be adjusted. The magnetic members 3306 may removably connect to the magnets 3370-1 (described below), so that the inferior side straps 3094 may be disconnected from the plenum chamber 3200, but the length of the inferior side straps 3094 may not be affected.

In some forms, the superior side straps 3092 may be connected directly to the tabs 3320 of the tubes 3350. The superior side straps 3092 may be threaded through the tabs 3320 in order to adjust the length and control the tensile force of each superior strap 3092.

### 5.3.3.2.2 Two-Point Connection

As shown in FIGS. 3C and 3D, some forms of the headgear 3302 may be a two-point connection headgear. This means that the headgear 3302 may connect to only two separate points on the patient interface 3000 i.e., one point on either side of the patient interface.

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 3C, the patient interface 3000 comprises a rear strap 3307 forming part of the positioning and stabilising structure 3300. The strap 3307 may be known as a back strap or a rear headgear strap, for example. The rear strap 3307 may overlay the temporal bones, parietal bone, and/or occipital bone. In other examples of the present technology, one or more further straps may be provided. For example, patient interfaces 3000 according to examples of the present technology having a nose-and-mouth cushion may have a second, lower, strap configured to lie against the patient's head proximate the patient's neck and/or against posterior surfaces of the patient's neck.

In the example shown in FIG. 3C, the rear strap 3307 of the positioning and stabilising structure 3300 is connected between the two tubes 3350 positioned on each side of the patient's head and passing around the back of the patient's head, for example overlying or lying inferior to the occipital bone of the patient's head in use. The rear strap 3307 connects to each tube above the patient's ears. With reference to FIG. 3C, the positioning and stabilising structure 3300 comprises a pair of tabs 3320. In use a rear strap 3307 may be connected between the tabs 3320. The rear strap 3307 may be sufficiently flexible to pass around the back of the patient's head and lie comfortably against the patient's head, even when under tension in use.

### 5.3.3.3 Rigidisers

In some examples of the technology, it can be advantageous for one or more of the straps of the positioning and stabilising structure to comprise a rigidiser 3340, as illustrated in FIG. 6B to 6G. A rigidiser 3340 may be an elongated, rigid, or substantially rigid member that adds rigidity, and/or reduces the elasticity/extensibility of the one or more straps of the positioning and stabilising structure. This can, in some examples, be used to assist in maintaining the seal-forming structure 3100 in an operating position on the face of the patient. For example, the rigidisers 3340 may be provided to the straps which contact a side of the patient's head and provide a force to limit slipping of the seal-forming structure 3100 from the patient's nose and/or mouth.

In some forms, the rigidiser 3340 is constructed from a material such as a polymer, or a metal. For example, the material may be substantially inextensible. The material may not permit the rigidiser 3340 to stretch. Additionally, the rigidiser 3340 may be substantially inflexible and may be unable to bend. The rigidiser 3340 may be pre-molded into a desired shape in order to fit a patient's head. For example, the rigidisers 3340 may be molded with a curved shape to substantially correspond to the shape of the side of the patient's head (e.g., overlaying the masseter muscle and/or the temporal bone).

In certain forms, the rigidiser 3340 may be molded in order to conform to a specific patient's head (e.g., the rigidiser 3340 is customized).

In some forms, the rigidiser 3340 may be flexible in at least one direction. For example, the rigidiser 3340 may be flexible about its width and may be inflexible along its length. In other words, the rigidiser 3340 may be bendable about an axis along the width of the rigidiser 3340 but may be unable to bend about an axis perpendicular to the rigidiser 3340. This may allow an individual patient to adjust the rigidiser 3340 in order to better fit their individual head.

The rigidisers are generally long and flat members having a length significantly greater than their width. In examples the rigidisers may also have a width significantly larger than their height, where the height is defined with respect to the distance the rigidiser extends upwardly, away from the surface of the patient's face in a direction substantially normal to the patient's face. This configuration can allow the rigidiser and associated strap to bend towards, or away from the patient's face in use, while remaining largely inflexible in other directions.

In certain forms, the rigidiser 3340 may remain in the new position after being bent. This may allow a patient to adjust the shape of the rigidiser 3340 for their specific head and then the rigidiser 3340 will keep the desired shape while in use in order to promote patient comfort.

In some examples the rigidiser 3340 may be attached to any one or more straps of a positioning and stabilising structure 3300, such as by adhesives, stitching, or any other suitable connection mechanism.

In some examples the rigidiser 3340 may be internal to the one or more straps. For example, the rigidiser may be positioned between one or more layers of the straps. In other examples, the rigidiser 3340 may be external to the one or more straps, in other words, the rigidiser may be at least partially external to the one or more straps. For example, at least a portion of the rigidiser may be positioned on a patient contacting side of the straps, or a non-patient contacting side of the straps. In some examples, the rigidiser may be substantially internal to the one or more straps, and comprise one or more sections which are external to the strap. For example, the rigidiser may weave in and out of the one or more straps; the one or more straps may comprise a window which exposes a portion of the rigidiser; and/or and end of the rigidiser may be exposed for example to facilitate connection of the top strap, rear strap or patient interface to the rigidiser.

In some examples of the technology, the rigidiser 3340 may be thermoformed, or otherwise moulded to the desired shape using a heat process, as should be familiar to those skilled in the art. For example, in forms of the technology comprising multi-layered straps, it may be beneficial for the rigidiser to be thermoformed as the various strap layers are brought together to form the strap. In other examples of the technology, the rigidiser 3340 may be a plastic or metallic insert which is positioned inside the headgear straps.

### 5.3.3.3.1 Construction

FIGS. 7A to 7J show cross-sectional views of rigidised strap constructions according to various forms of the present technology. As shown in FIG. 7A, the rigidised strap may include a rigidiser 3340 that is attached to one or more strap layers 7002, for example the strap layers 7002 may comprise a fabric, textile or foam. In examples of the technology the strap layers 7002 may be configured to provide a patient contacting portion or side 7004, and a non-patient contacting portion or side 7006, wherein the patient contacting portion 7004 is substantially opposite to the non-patient contacting portion 7006, for example the non-patient contacting portion 7006 may be configured to face outwardly from the patient's head in use, while the patient contacting portion 7004 may be configured to face inwardly, so as to contact the skin or hair of the patient 1000 in use.

In some examples of the technology, the non-patient contacting portion 7006 of the positioning and stabilising structure may comprise an unbroken loop material such as a fabric, for example the unbroken loop material may be selected to be compatible with hook and loop fasteners. In one example, the unbroken loop material may be used across all of the non-patient contacting portions 7006 of the positioning and stabilising structure. One potential advantage of this arrangement is that it allows for a single material to be used on the non-patient contacting portion 7006. In other examples, the unbroken loop material may be selectively provided in the one or more side straps, such as the superior 3092 and inferior 3094 side straps, one or more top straps 3096, and one or more rear straps 3307 as described herein.

In some forms of the technology, the rigidised strap may comprise a single strap layer 7002 which encases or otherwise attaches to the rigidiser. For example, the strap layers 7002 may be configured to extend around the rigidiser 3340 from a patient contacting portion 7004 to a non-patient contacting portion 7006 as shown in FIGS. 7B, 7E, and 7J. In other forms of the technology, the rigidised strap may comprise two or more strap layers 7002 which are configured to provide a patient contacting side, and a non-patient contacting side, as shown in FIGS. 7A, 7C, and 7G. For example, the two or more strap layers 7002 may be joined using any techniques known to those skilled in the art, including for example by stitching, gluing or ultrasonic welding.

Where the strap layers 7002 are joined, a joint 7008 may be formed. In some examples of the technology, it may be preferable for the joint 7008 to be configured such that it is not located in a patient contacting surface of the strap, as this could potentially cause discomfort or facial marking of the patient in use. In some examples it may be advantageous for the joint 7008 not to be visible in the non-patient contacting side of the strap, as it may be considered aesthetically unappealing.

Accordingly, one aspect of the present technology is to provide a rigidised strap, wherein the joint 7008 does not contact the patient's face in use. This may be achieved by sewing the one or more strap layers 7002 inside out and returning it to its intended orientation so that the stitching is within the pocket of the strap layers 7002. The strap layers 7002 provides a softer surface for contacting the patient's face in use.

In some examples, the strap layer(s) 7002 on the patient contacting side 7004 may be the same as the strap layer(s) 7002 on the non-patient contacting side 7006. The strap layer(s) 7002 on the patient contacting side 7004 may preferably have the same weave as the strap layer(s) 7002 on the non-patient contacting side 7006, such that the stretch characteristics of the straps are approximately equal on both sides. In some examples it may be preferred that the strap layer(s) 7002 on the patient contacting side 7004 have the same heat shrinkage characteristics as the non-patient contacting side 7006. This is to prevent the headgear deforming unevenly when thermoformed or otherwise processed or exposed to heat. The strap layer(s) 7002 on the patient contacting side 7004 may be a different fabric to the non-patient contacting side 7006, such that the strap layer(s) 7002 on the patient contacting side 7004 are more comfortable than the non-patient contacting side 7006.

As shown in FIG. 7B, the rigidised strap may include a rigidiser 3340 for example a rigidiser formed from or otherwise cut from a sheet of rigidiser material, as will be discussed in more detail in respect of FIG. 8A. The rigidiser may be overmolded with a soft polymeric material to provide the strap layer(s) 7002. For example, the polymeric material may be a thermoplastic elastomer (TPE), or thermoplastic polyurethane (TPU). The polymeric material may in some examples provide a soft material for contacting the patient's face in use.

As shown in FIG. 7C, the rigidised strap may include a semi-rigid moulded rigidiser 3340 that is covered in a fabric strap layers 7002 e.g., two pieces of fabric joined by stitching.

As shown in FIG. 7D, the rigidised strap may include a semi-rigid moulded rigidiser 3340 that is attached (e.g., welded, glued, overmolded) to a fabric composite strap layer 7002. For example, a material such as a neoprene, or such as is sold under the Breath-O-Prene™ trademark may be used. The fabric composite strap layer 7002 may provide a softer material for contacting the patient's face in use.

As shown in FIG. 7E, the rigidised strap may include a semi-rigid moulded rigidiser 3340 that is overmoulded with a strap layer 7002 in the form of a soft polymeric material such as TPE, or TPU. The polymeric material may provide a softer material for contacting the patient's face in use.

As shown in FIG. 7F, the rigidised strap may include a soft moulded rigidiser 3340 moulded with a soft polymeric material strap layer 7002, e.g., TPE, TPU. In an embodiment, the moulded component may be provided with soft touch or flock coating.

As shown in FIG. 7G, the rigidiser may include a strap layer 7002 which is thermoformed and attached to the rigidiser 3340. The rigidiser 3340 may be formed from a sheet of material, such as by die cutting. In other examples, the rigidiser could be a moulded part, machined part, or otherwise formed part. The fabric outer strap layers 7002 may be heat sealed together, stitched, ultrasonically cut, CNC knife cut, or otherwise joined. As illustrated, the fabric outer layer is joined at approximately the centre or middle of the edge. Preferably, the joint 7008 is at the centre or close to the centre of the side of the headgear.

Additionally, in the examples described herein, the joint may be positioned away from the patient's face when in use. For example, the edges of the patient contacting portion 7004, may be rounded, or otherwise curved upwardly, away from the face of the user to improve the comfort of the rigidised straps on the head of the patient 1000. For example, it may be desirable for one or more edges of the straps to be curved outwardly of the patient's face in one or more sensitive areas of the patient's head, such as around the ears of the user, or around the rear of the patient's head. For example, with respect to FIG. 8A, in some examples of the technology, it can be advantageous for the positioning and stabilising structure to include a soft-edge (i.e. an edge formed using a soft cushioning fabric or foam) or a outwardly curved edge in the region adjacent to the ear of the patient, such as in a region 8002 immediately superior to, and/or posterior to the ear of the patient. While this region is described in respect of the positioning and stabilising structure, it should be appreciated that in examples of the technology, a rigidiser may also be provided in this region 8002. As such, this region 8002 may be described as an arcuate region with a curvature which connects a first end 5012 of a rigidiser 3340 to a second end 5014 of the rigidiser. For example, the region 8002 may connect part of the rigidiser which is anterior of the patient's ear, to a part of the rigidiser which is posterior to the patient's ear by connecting in a region which is superior to the patient's ear.

In some examples a layer of foam 7010 or other conformable material may be positioned around or about the rigidiser 3340. The use of a foam such as a memory foam, open cell foam or closed cell foam, such as polyurethane may advantageously improve the comfort of the straps, particularly in regions comprising the rigidiser. The foam 7010 may preferably extend to the lateral, horizontal edges of the rigidiser 3340 so as to prevent the ends of the strap layers 7002 from abutting the patient's face and causing discomfort or facial marking. For example, FIG. 7H illustrates a rigidised strap with fabric outer strap layers 7002 and a foam layer 7010 provided along at least a portion of the rigidiser 3340. As illustrated, the foam layer 7010 positions the joints 7008 away from the patient's face when in use.

In an embodiment, the headgear strap may be thermoformed and then edges of the strap may be cut, for example using a die cut, a CNC controlled cutter, or by an ultrasonic cutting process. In some forms of the technology is may be advantageous to use an ultrasonic cutting process in order to provide rounded edges, which can provide increased comfort and reduced facial marking in use. In addition, thermoformed and ultrasonically cut edges have been found to be softer and less abrasive, which provides a more comfortable feel on the patent's face in use, e.g., more comfortable feel around the patient's ears.

In a further embodiment, at least a portion of the headgear may be constructed from a spacer fabric, where the edges of the spacer fabric may be ultrasonically welded. This may cause the edges of the spacer fabric to be rounded, thereby reducing facial marking, and increasing comfort for the patient.

In an alternative embodiment, the fabric outer layers may be attached together by adhesive. For example, as shown in FIG. 7I, a first layer of fabric 7002a may have wings 7012 positioned at one or both ends. A joint 7008 may be formed using an Adhesive (e.g., glue). The adhesive on ends of a second layer of fabric, such that the wings can be folded onto the adhesive to seal the inner portions of the headgear within the fabric layers. As illustrated, the inner portions of the headgear may include a conformable material such as foam, or a rigidiser 1580, or a combination of the two. Alternative internal components may include other elements disclosed herein, such as three-dimensional weaves. It should further be appreciated that the adhesive and wings may be positioned in alternative arrangements, such as the adhesive positioned on the wings, or the wings positioned on the second layer of fabric. It is further possible for the first and second layers of fabric to be a single, continuous piece of fabric that is sealed at one end by a wing and adhesive arrangement. For example, FIG. 7J shows a single piece of fabric including a wing at one end that is adapted to be folded onto adhesive (e.g., glue) at the other end. As illustrated, the inner portions of the headgear include a conformable material such as foam and a rigidiser 3340.

Preferably, the edges of the headgear are completely closed, that is, the interior components of the headgear (such as the rigidiser and conformable material) are completely contained within the fabric outer layers. This is to avoid hair tangling in the internal components or discomfort due to contact with the interior elements. In addition, it may be easier to maintain the cleanliness and durability of the system if the internal components are completely encapsulated or contained within the fabric layers.

Preferably, the edges where the fabric layers meet one another are sealed or otherwise hidden to avoid the fabrics from parting or becoming dislodged. This arrangement may also be preferable for creating a rounded continuous edge. See FIG. 7A for example.

In an embodiment, the rigidiser may be relatively thin, e.g., less than 1 mm such as 0.5 mm or 0.8 mm. In another example, the strap may include a nylon rigidiser enclosed in foam. In such embodiment, the density of the foam may be increased to improve comfort and reduce chances of feeling the nylon rigidiser. Alternatively, the thickness of the foam may be utilized to alter the softness or roundness of the edge of the headgear. For example, thicker layers of foam are more likely to produce rounder corners than thinner layers of foam. In a further embodiment, the foam may begin at one thickness, and be compressed to another thickness during processing. The first thickness of the foam may be 5 to 30 mm. Preferably, the first thickness may be 7 to 12 mm. Alternatively, the first foam thickness may be 10 to 20 mm. The second foam thickness may be 0.1 to 10 mm. Preferably, the second foam thickness may be 2 to 5 mm. The second foam thickness may alternatively be 3 to 7 mm.

In some examples of the technology, the rigidiser 3340 may be formed by a heat pressing or embossing process configured to change the properties, such as the rigidity of a foam inner to thereby melt the foam and create a stiffened region. For example, a rigidiser may be formed by compressing or thermoforming to reduce the thickness of the foam by between 50 and 95%, such as approximately 80%.

In some examples the rigidiser 3340 may include embossed ribs or other features to encourage flex or control movement of the headgear in specific regions. In addition, embossing may be used to stamp on a branding logo. In other examples, the rigidiser may comprise two or more limbs which provide a gap therebetween, as is described in relation to FIGS. 8C and 8D.

In an example, the headgear strap may include two layers of foam. However, other suitable configurations are possible, such as a one layer or three or more layers of foam. In an embodiment, foam on the patient contacting side may be less dense or have a lower hardness than foam on non-patient contacting side. It is also possible to have more than one layer of foam and more than one rigidiser component. Alternatively, the headgear may comprise more than one rigidiser and a single layer of foam.

In an example, non-woven material may be inserted in-between additional foam or fabric layers in place of a nylon rigidiser.

In an embodiment, the materials and/or headgear configuration may be selected to reduce costs.

In certain forms of the technology, it may be advantageous for the positioning and stabilising structure 3300 to comprise varying thicknesses. For example, it may be advantageous to add thickness to one or more of the straps to provide additional cushioning or comfort in certain regions. In other examples of the technology, it may be advantageous to minimise the total material thickness in certain regions, for example for users who are side-sleepers it may be advantageous for the material thickness in the side of head regions to be less than the material thickness on top of the user's head, or in the cheek regions, and similarly for users who are typically back sleepers, it may be advantageous for the material thickness in the regions of the positioning and stabilising structure 3300 on the rear of the user's head to be less than to be the material thickness on top of the user's head, or in the cheek regions.

In examples of the technology illustrated in FIG. 8A the side straps, of the positioning and stabilising structure 3300 may comprise a rigidised portion 3300A comprising a rigidiser 3340 (not shown in FIG. 8A). The rigidised portion may be configured to connect to one or more side straps, such as the superior 3092 and inferior 3094 side straps, one or more top straps 3096, and one or more rear straps 3307. For example, each of the foregoing portions/straps may be integrally formed, such as by cutting from a single sheet of material such as a fabric or foam (or a laminated stack of materials), or alternatively any one or more of the straps/portions may be joined for example using adhesives, stitching or ultrasonic welding as described herein.

In examples, the rigidised portion 3300A may have a thickness of between approximately 7 mm and approximately 3 mm, such as substantially 5 mm. In examples it may be advantageous for the side straps such as the superior 3092 or inferior 3094 side straps, the top strap 3096, and/or the rear strap 3307 to be thinner or have a lower thickness than the rigidised portion 3300A. For example, side straps such as the superior 3092 or inferior 3094 side straps may have a thickness of between approximately 4 mm and approximately 1 mm such as 2.7 mm, while the top strap 3096 and/or rear straps 3307 may have a thickness of between approximately 3 mm and approximately 6 mm such as substantially 5 mm.

In some examples it may be advantageous for the rigidised portion to have a thickness which is as thin as possible while maintaining the desired in-plane stiffness required for supporting the patient interface 3000 in position on the patient's head. For example, it may be desirable for the rigidised portion to have a thickness of less than 6 mm such as between 3 mm and 5 mm. The use of a thin rigidised portion may advantageously improve comfort for side-sleepers.

In some examples of the technology, such as shown in FIG. 8B, it may be desirable for a rigidiser 3340 to be covered with a sleeve 8008, or other piece of cushioning material which can further increase the comfort, and/or reducing the likelihood of facial marking. For example, the sleeve 8008 may be configured to wrap around one or more of the headgear straps, or alternatively, the sleeve may comprise a tubular construction which is configured to be slid over one or more of the headgear straps in use. In some forms of the technology, the sleeve may be removable, for example to facilitate ease of cleaning.

### 5.3.3.3.1.1 Rigidiser Limbs

In some examples of the technology, such as in the embodiment shown in FIG. 8C, it may be beneficial for the rigidiser 3340 to comprise two or more limbs or branches 8004 which each extend along substantially the same longitudinal section of the headgear straps. For example, in FIG. 8C a first pair of limbs 8004A, 8004B extend superior to the ear of the patient in use, towards the top strap 3096, or connection member 3098 configured to connect to a top strap 3096. A second pair of limbs 8004C, 8004D are provided in a region of the straps which is posterior to the ear of the patient in use, in a portion of the strap which extends towards the rear strap 3307.

In other words, the first limb 8004A and second limb 8004B may be offset from one another along a longitudinal portion of the at least one strap (e.g., laterally offset from one another such that both limbs occupy the same longitudinal section of the strap). In some examples the limbs are spaced apart from one another so as to provide a space therebetween. In some examples the limbs 8004 may be substantially parallel to one another, in other examples, such as shown in FIG. 8C the limbs may either converge or diverge from one another. In the examples shown the distal ends of the first 8004A and second 8004 limbs, i.e., the ends of the limbs which are furthest from a centre of the rigidiser, are connected to one another, for example, either by converging in the case of the superior limbs 8004A, 8004B, or by being connected via a bridging section 8006 in the example of the posterior limbs 8004C, 8004D. In other examples of the technology, such as shown in FIG. 8D, the distal ends of each of the limbs may have a substantially free end, or otherwise be separated from one another which may allow for reduced torsional rigidity in comparison to the example of FIG. 8C.

The use of converging or diverging limbs may advantageously allow the torsional stiffness or rigidity of the one or more straps to be adjusted along their length by selectively controlling the amount out-of-plane bending each limb may be able to do relative to the other of the first and second limbs.

Conversely, to increase the rigidity in portions of the one or more straps, the width of the rigidiser may be increased. For example, as illustrated, it may be desirable to have a wider rigidiser in a region which is anterior of the patient's ear in use. For example the region of the rigidiser anterior of the patient's ear may be configured to connect to one or more straps (such as the superior 3092 and inferior straps 3094) for the purposes of tightening the patient interface on the patient's face.

The use of rigidiser limbs 8004, may also advantageously provide for a better connection between the straps and the rigidiser material, as a join between the inner, patient contacting layer, and outer-non patient contacting layer may be made through the gap formed between each of the limbs. In some examples of the technology, this gap may be filled, for example using an additional section of material (such as rigidiser material, foam or a textile) such that the difference is thickness is less pronounced, this may improve the aesthetics, and/or the comfort of the positioning and stabilising structures described herein. In some examples, it may be advantageous for the gap between the rigidiser limbs to be filled with a foam material, which has a greater thickness than the rigidiser, so as to lifts the rigidiser outwardly away from the patient's skin, further improving comfort, and/or reducing facial marking.

It should be appreciated that the use of rigidiser limbs in the specific locations illustrated in FIGS. 8C and 8D are not limiting on the scope of the technology, and these features may be added in any rigidiser construction where it may be advantageous to increase the torsional flexibility, or otherwise reduce the out-of-plane rigidity of the headgear straps. For, example, limbs could be used in any portion of the rigidiser 3340 construction illustrated in FIG. 6E, such as in the region superior to the ear of the user to reduce the torsional rigidity in this region.

### 5.3.3.3.2 Manufacturing Techniques

In one form of the technology, the positioning and stabilising structures 3300 described herein may comprise a first rigidiser 3340A, configured for use on a first side of the patient's head and a second rigidiser 3340B, configured for use on a second side of the patient's head. In some examples the first rigidiser 3340A may comprise a shape which has at least one axis of symmetry with the second rigidiser 3340B, as shown in FIG. 9A, such as the first rigidiser being a mirror image of the second rigidiser. For example, it may be advantageous to have a single stock of rigidisers used in the construction of the rigidised positioning and stabilising structures, for example, a first rigidiser may be flipped over in order to be used in the manufacture of headgear straps for the opposite side of the patient's head.

In some forms of the technology, the rigidiser 3340 may be cut from a sheet of rigidising material, such as a sheet of thin plastic or metallic material. For example, the rigidiser may be formed from a sheet of nylon. In certain forms of the technology, it can be advantageous for the rigidiser to be resiliently deformable, such that it substantially regains its shape after undergoing deformation.

In some examples of the technology, it can be advantageous for the rigidiser to be formed of a continuous piece of material, for example to reduce the manufacturing costs associated with joining pieces of material. In other examples of the technology, the rigidiser 3340 may comprise a plurality of rigidiser sections which are joined for example such as by welding, stitching or adhering the layers together or to layers of the headgear straps. For example, by providing discrete rigidiser components it may be possible to improve material efficiency by increasing the number of rigidiser components which can be produced from a single sheet of rigidiser material.

In examples of the technology comprising rigidiser limbs 8004, such as FIG. 8C, and 8D, the corresponding gaps between in each limb may be formed using any techniques known to those skilled in the art, including die-cutting, stamping, cutting etc.

In some forms of the technology, it can be advantageous for the rigidiser 3340 to have a first stiffness bending in-plane, which is greater than its bending stiffness out-of-plane. Examples of being in-plane are shown in FIG. 9B, and out-of-plane illustrated in FIG. 9C. In general terms, examples of the technology may comprise rigidisers 3340 formed from substantially planar sheet materials, or otherwise the rigidised straps once formed, may be considered to have a substantially planar construction when formed. Accordingly, bending in-plane may be defined as bending which occurs around an axis which is substantially perpendicular to the plane of the rigidiser/rigidised straps, while being out of plane may be defined as bending which occurs around an axis which is substantially parallel to the plane of the rigidiser/rigidised straps.

For example, by having a high in-plane stiffness or rigidity, the positioning and stabilising structure may be better able to support and resist any superior-inferior movement of the patient interface on the face of the user, by transferring the associated forces through the rigidiser to the rear of the user's head. Conversely, by having a lower out-of-plane stiffness or rigidity, the positioning and stabilising structure may be able to better conform to the shape of the patient's facial features, such as the bone structure of their face.

### 5.3.3.4 Over-the-Ear Headgear

In examples of the technology, such as in FIG. 3D nasal interfaces may be configured such that the positioning and stabilising structure 3300, extends in use from the patient interface 3000 along a side of the patients face, for example in a direction overlaying the cheekbones of the patient superior to the otobasion superior. In other words, one or more straps may be configured to extend above the ears of the patient, to connect to the top strap 3096 and rear strap 3307. This configuration may be beneficial to the patient 1000 as the patient interface 3000 and associated positioning and stabilising structure 3300 may be put on (“donned”) and/or taken off (“doffed”) without needing to connect, disconnect or adjust any of the straps between use, and/or without needing to connect a lower headgear strap 3094 inferior to the ear of the patient.

In nasal interfaces, this can be achieved as the stabilising (F_{PSS}) forces required to counteract the blowout forces (Fₚₗₑₙᵤₘ) can be provided in a direction which extends above the ear of the patient in use. For example, as illustrated in FIG. 3D, the stabilising forces F_{PSS} may be provided along an axis x, having an angle a which is between approximately 35 degrees with respect to the Frankfort horizontal, and approximately 25 degrees with respect to the Frankfort horizontal, such as approximately 30 degrees. In nasal interfaces this angle has been shown to be effective, as the resulting force FPSS can help to force the seal-forming structure 3100 in a superior direction into engagement with or around the nares of the patient.

In a patient interface 3000, which comprises a mouth seal and a nose seal however, the Plenum forces Fₚₗₑₙᵤₘ may include a larger component in the axis of the Frankfort Horizonal, for example due to inflation of the seal forming structure against any one or more of the chin, lower lip region, upper lip regions, and nasal bridge regions of the patient face. Accordingly, the blowout forces Fₚₗₑₙᵤₘ are at a lower angle a with respect to the Frankfort horizontal, and therefore the use of traditional two-point headgear arrangements is not possible without the straps of the headgear extending over the ears of the patient 1000 in use which can result in discomfort for the patient, particularly when the patient is in sleeping on their side.

### 5.3.3.4.1 Four-Point Over-the-Ear Headgear

FIG. 5A shows one example of the technology, where a four-point positioning and stabilising structure 3300 is provided to support a patient interface 3000 on a patient's face. In other words the positioning and stabilising structure attaches to the patient interface at four-points, two on either side of the patient interface. It should be appreciated that reference here to two or four-point positioning and stabilising structures 3300 should not be seen as limiting on the technology and any number of connection points may be provided, including three or more connection points on each side of the patient interface.

In the illustrated example the positioning and stabilising structure 3300 comprises a superior side strap 3092, which connects via a connection member 3098 to a superior portion of the patient interface, and an inferior side strap 3094 which connects via a connection member 3098 to an inferior portion of the patient interface.

The superior strap 3092 and inferior strap 3094 may be independently formed straps which are joined using any method known to those skilled in the art, including stitching, and ultrasonic welding. Alternatively, the superior strap 3092 may be integrally formed with the inferior strap 3094, for example a single strap may be split to form the superior strap 3092 and inferior strap 3094.

In the example of FIG. 5A the top strap 3096 and rear strap 3307, are further joined by a connecting strap 5002. In some forms, the connecting strap 5002 may take the form of webbing. For example, the connecting strap may comprise an elastic or mesh material which allows for flexibility between the top strap 3096 and the rear strap 3307. In other forms, the connecting strap 5002 may be another strap, or a panel of material, for example a gusset.

In use the tension in the superior strap 3092 may be adjusted to adjust the F_{PSS} force applied to a superior portion of the patient interface. For example, the superior strap 3092 may be shortened to increase the sealing pressure in a region adjacent to the nose of the patient 1000, or conversely lengthened to reduce the sealing pressure in this region. For example, the sealing pressure may be in the nasal bridge region, side of nose regions or upper lip regions.

In use the tension in the inferior strap 3094 may be adjusted to adjust the F_{PSS} force applied to an inferior portion of the patient interface. For example, the inferior strap 3094 may be shortened to increase the sealing pressure in a region adjacent to the chin or mouth of the patient 1000, or conversely lengthened to reduce the sealing pressure in this region. For example, the sealing pressure may be in the chin, side of mouth or lower lip regions of the patient's face.

In order to provide a different F_{PSS} force vector in each of the superior and anterior straps, it may be beneficial to provide different anchor points for each of the respective superior 3092 and inferior 3094 straps. For example, the inferior strap 3094 may be configured to extend from the patient interface, along the side of the face of the patient from an anterior side of the patient's ear, superior to the patient's ear, to a posterior side of the patient's ear to join with or otherwise form the rear strap 3307. In this way, the inferior strap 3094, follows an arcuate path, around the ear of the patient in a substantially inverted U-shape.

Accordingly, the inferior strap 3094, as measured from the connection point on the patient interface through to the anchoring point behind or otherwise posterior to the patient's ear, provides a stabilising force F_{PSS} having an angle α with respect to the Frankfort horizontal, such as between approximately 0 degrees, and approximately 20 degrees, such as approximately 10 degrees. In some examples of the technology as described here it may be beneficial for the inferior strap 3094 to be constructed of a material which has a greater stiffness, or otherwise lower flexibility than the superior strap. In some examples of the technology, the rigidity of the inferior strap 3094 may be increased by using one or more rigidisers as described herein.

The superior strap 3092 may extend from the patient interface along the side of the face of the patient to a region substantially superior to the ear of the patient before joining with, or otherwise forming the top strap 3096. Accordingly, the superior strap 3092 may provide a stabilising force F_{PSS} along an axis ‘y’ having an angle β with respect to the Frankfort horizontal, such as between approximately 25 degrees, and approximately 35 degrees, such as approximately 30 degrees.

### 5.3.3.4.2 Two-Point Over-the-Ear Headgear

FIG. 5B shows one example of the technology, where a two-point positioning and stabilising structure 3300 is provided to support a patient interface 3000 on a patient's face. In other words, the positioning and stabilising structure attaches to the patient interface at two-points, one either side of the patient interface. In the illustrated example the positioning and stabilising structure 3300 comprises a side strap 5004, which connects via a connection member 3098 to the patient interface.

As in the previous examples of the technology, the two-point positioning and stabilising structure 3300 comprises a top strap 3096, and a rear strap 3307 as described herein.

The two-point positioning and stabilising structure 3300 comprises a side strap 5004, which functions in a similar manner to the inferior side straps of the four-point headgear examples in that the side strap 5004 is configured to provide an angle α between the point of connection to the patient interface, and the opposing end of the side strap, posterior to the ear of the patient. The angle α providing a stabilising force F_{PSS} having angle α with respect to the Frankfort horizontal, such as between approximately 0 degrees, and approximately 20 degrees, such as approximately 10 degrees.

### 5.3.3.4.3 Rigidised Side Straps

FIG. 5C shows a further example of a positioning and stabilising structure 3300 in accordance with the present technology. In the illustrated example a single side strap 5004 is provided on each side of the patient interface to provide a two-point headgear configuration. The side strap 5004 extends from a region anterior to the patient's ear, extending first superior to the patient's octobasion superior, and continuing downwardly to a region posterior of the patient's ear, before connecting to the rear strap 3307.

In this example the side strap 5004 is provided with a rigidiser 3340 to improve the rigidity of the side strap, and to more directly transfer the forces imparted to the patient interface end of the side strap 5004, through to the top strap 3096, and rear strap 3307. In some examples, the rigidiser may also help to anchor the headgear using the stable bony parts of the cranium superior to and in some cases posterior to the ears of the patient.

The rigidiser 3340 may be provided to the side strap 5004 in any suitable manner, such as by being affixed to an outer surface of the side strap 5004 facing away from the patient in use, or embedded in the side strap, i.e., positioned between one or more layers in the side strap.

In the illustrated example, the side strap 5004 and rigidiser 3340 have a substantially inverted Y-shape, having a first end 5006 adjacent to, and configured to connect to the patient interface 3000, and a second end 5008 which is distal to the first end and is configured to connect to the rear strap 3307. In the illustrated example the side strap 5004 also extends upwardly toward the top strap to a third end 5010 which is positioned in a region which is superior to the patient's ear.

Having the rigidiser extend upwardly from the otobasion superior may raise the anchoring point of the positioning and stabilising structure, relative to rigidisers without the upward extension. The higher anchoring point, may for some patients provide a more stable positioning of the patient interface.

In the illustrated example, the rigidiser 3340 comprises a first end 5012 on an anterior side of the patient's ear, and a second end 5014 on a posterior side of the patient's ear. In use forces imparted to the first end 5012 by the patient interface are transferred to the second end 5014. Accordingly, by anchoring the second end 5014 of the rigidiser to the rear strap, the first end 5012 becomes similarly anchored.

In examples of the technology, it can be advantageous for the first end 5012 of the rigidiser to be positioned such that it overlays or is otherwise positioned inferior to the cheekbones of the patient. In other words, it can be advantageous for the angle between the point at which the side strap 5004 connects to the patient interface, and the first end of the rigidiser to be less than 25 degrees with respect to the Frankfort horizontal.

In other examples, the rigidiser 3340 may be defined by an axis ‘x’ which extends between the first end 5012 of the rigidiser 3340 and the second end 5014 of the rigidiser 3340, wherein it may be advantageous for the angle ‘α’ of the axis ‘x’ with respect to the Frankfort horizontal may be less than 25 degrees.

In some examples of the technology, the side straps 5004 are configured to attach to the patient interface 3000, top strap 3096, and rear strap 3307 by extending portions of the strap through connection members 3098 in the respective patient interface 3000, top strap 3096 and rear strap 3307. This configuration should not be seen as limiting and in the illustrated example the side strap 5004 comprises connection members 3096 configured to receive the respective top 3096 and rear straps 3307.

FIG. 5D shows a further example of the present technology, where a side strap 5004 splits into a plurality of side straps in the form of a superior strap 3092, and an inferior strap 3094. A rigidiser 3340 is provided to the inferior strap 3094 to further add rigidity to the inferior strap.

Referring to FIGS. 6A to 6G, there is provided a number of rigidiser 3340 examples according to the present technology.

FIG. 6A shows a first example of a positioning and stabilising structure comprising a rigidiser 3340 in the form of a rigid strap. In other words, the inferior strap 3094 may be comprised of a material such as a polymer which provides the strap with the necessary rigidity to transfer the forces imparted to the patient interface through to the rear strap 3307.

In each of the foregoing examples, the first end 5012 of the rigidiser 3340 is illustrated as being positioned on the side strap in a location which is set back from the patient interface. This configuration may advantageously allow for adjustment between the side straps and the patient interface, including both tension adjustment and angle adjustments. However this should not be seen as limiting and in other examples the rigidiser may be configured to connect directly to the patient interface 3000.

FIG. 6B shows an example of a positioning and stabilising structure 3300 which comprises a rigidiser 3340 having a first end positioned such that it overlays or otherwise sits inferior to the cheekbones of the patient, and anterior of the ear of the patient 1000. The rigidiser extends along the contour of the side strap 5004 following the curvature of the strap above or otherwise superior to the ear of the user, and having a second end which is superior to, and slightly posterior to the ear of the patient.

FIG. 6C shows a modified version of the positioning and stabilising structure 3300 of FIG. 6B in which a second rigidiser 3340 is provided in a location which is superior to the ear of the patient, and extends upwardly, to partially follow the direction of the top strap 3096. In this way, both rigidisers may function to support and anchor the first end of the rigidiser in a location which sits on or below the cheekbones of the user. Note that in this example each of the rigidisers may be separate to one another, by leaving a small gap between the rigidisers, it may be possible to adjust the amount of movement allowed in the patient interface 3000 to improve patient comfort in use.

FIG. 6D shows a further example of a rigidiser in which the rigidiser 3340 has a first end 5012, positioned such that it overlays or otherwise sits inferior to the cheekbones of the patient, and anterior of the ear of the patient 1000. The rigidiser extends along the contour of the side strap 5004 following the curvature of the strap above or otherwise superior to the ear of the user, and having a second end which is superior to, and slightly posterior to the ear of the patient. In this example the rigidiser also extends upwardly, in a direction superior to the ear of the patient such that it substantially follows the direction of the top strap. In this way, the rigidiser may be better anchored horizontally by the rear strap, and vertically by the top strap.

FIGS. 6E to 6G largely correspond to the examples of FIGS. 6B to 6D, with the exception that the second end of the rigidiser is configured to extend further along the rear strap, posterior to the ear of the user. For example, the second end of the rigidiser may be configured to use overlay a point where the parietal bone of the patient's head meets the temporal bone, or otherwise posterior to a point where the parietal bone of the patient's head meets the temporal bone. In other examples of the technology, the second end of the rigidiser may be configured to use overlay a point where the occipital bone of the patient's head meets the temporal bone, or otherwise a point which is superior to the point where the occipital and temporal bones meet.

In other examples the rigidiser may be defined as extending in an arcuate path centred around the ear of the user, wherein the arcuate path has an arc angle of between approximately 90 and approximately 145 degrees, such as approximately 130 degrees (in the examples of FIG. 6B to 6D). In other examples the rigidiser may be defined as extending in an arcuate path centred around the ear of the user, wherein the arcuate path has an arc angle of between approximately 170 and approximately 210 degrees, such as approximately 180 degrees (in the examples of FIG. 6E to 6G).

By extending the rigidiser length, and/or the associated arc angle, the corresponding angle between the first end 5012 of the rigidiser and the second end 5014 of the rigidiser is reduced relative to the Frankfort horizontal. Thereby making the resulting support force vector FPSS which is substantially horizontal, or otherwise closer to the Frankfort horizontal. This may be beneficial for example when used in full-face masks which seal with the nasal bridge region of the patient, in order to minimise the pressure on the sensitive nasal bridge region, while maintaining an effective seal.

### 5.3.3.5 Headgear Movement During Use

In use positioning and stabilising structures 3300 can move from their intended position on the patient's 1000 face, thereby affecting the comfort and efficacy of the pressure therapy being provided. This effect can be exacerbated in examples of the technology where there are relatively few anchoring points for the patient interface 3000 on the patient's head.

FIG. 10A shows one example of a rigidiser 3340 in position on a patient's 1000 head. For sake of clarity the straps of the headgear have been omitted from this example, however details of these straps are substantially the same as has been described herein.

In normal use the rigidiser experiences three main force vectors: a sealing force (Fₛ) which is the force which is pulling the patient interface 3000 into engagement with the patient's face, and two headgear forces (F_{H1} and F_{H2}) which oppose the sealing force Fₛ.

In the illustrated example, two sealing force (Fₛ) vectors are shown, which correspond to two attachment points between the rigidiser 3340 and the patient interface 3000 (i.e., the superior strap 3092 and inferior strap 3094 of, for example FIG. 6G) although this should not be seen as limiting and in other examples a single side strap 5004 (of for example, FIG. 6A) may be used between the patient interface 3000 and the rigidiser 3340.

In use, tightening the straps increases the sealing force Fₛ, while simultaneously increasing the forces (F_{H1} and F_{H2}) that are transferred through the rigidiser to the top and rear of the positioning and stabilising structure 3300, such as at the rear strap 3307 and top strap 3096.

While the rigidiser is configured to have a relatively high in-plane stiffness, these forces may result in movement or translation of the first end 5012 of the rigidiser 3340. Additionally, the headgear may pivot slightly on the head of the user, in response to these forces. Indicated in FIG. 10A is an example of a pivot point 10002 for a headgear which uses a rigidiser as shown.

It should be appreciated that references to the pivot point 10002 in the specification are in reference to a point about which the first end 5012 of the rigidiser pivots in use such as when the headgear is tightened. This pivot point 10002 is discussed with reference to side views of the rigidiser 3340 structure, which substantially sits within a two-dimensional plane. Accordingly, when considering this two-dimensional perspective, the pivot point 10002 acts as the centre or focus of an imaginary circle having a radius equal to the separation between the first end 5012 and the pivot point, and wherein movement of the first end 5012 in use follows the circumference of the imaginary circle.

It should be appreciated however that the headgear is a three-dimensional structure, and as such, when donned by a patient, the pivot points 10002 on opposing sides of the headgear provide a pivot axis around which the respective first ends 5012 pivot in use.

As shown the pivot point 10002 is positioned superior and slightly posterior to the ear of the patient 1000. However the location of the pivot point 10002 may vary slightly depending on the anthropomorphic characteristics of the patient, and the associated sizing of the positioning and stabilising structure 3300.

Accordingly, during tightening of the headgear, the first end 5012 of the rigidiser 3340 may translate on or about an arc ‘A’ which is substantially defined by the location of the pivot point 10002; in other words, the first end 5012 of the rigidiser may translate in an anterior-superior direction, i.e., towards the nose and/or eyes of the patient 1000. This translation of the rigidiser 3340 can have a negative impact on the sealing performance of the patient interface 3000 or otherwise decrease the comfort of the system. For example, as the first end 5012 of the rigidiser translates upwardly, or in a superior direction on the patient's face, the resulting sealing force vectors Fs may encourage the patient interface 3000 upwardly in a superior direction on the patient's face, thereby increasing the forces imparted into the patient's nose, potentially reducing comfort, and/or sealing efficacy.

In light of this, a further example of the technology shown in FIGS. 10B and 10C provides a version of the rigidiser 3340 configured with a third end 5010 which extends higher on the head of the patient, in a direction superior to the ear of the patient 1000. For example, the third end 5010 may be positioned between approximately 60 mm and approximately 120 mm (inclusive) above or superior to the ear of the patient, such as between approximately 80 mm and approximately 100 mm inclusive. By providing a rigidiser 3340 which has a third end 5010 which sits higher on the head of the patient in use, the resulting pivot point 10002 for the rigidiser 3340 also moves higher on the patient.

This has the added effect of increasing the separation between the pivot point 10002 and the first end 5012, and accordingly increasing the radial distance of the arc ‘A’ from the pivot point 10002. As such, during translation of the first end 5012, such as due to tightening of the headgear, the first end 5012 travels in a substantially posterior to anterior direction and has less movement in an inferior to superior direction on the patient's face.

In the illustrated examples the region 8002 of the positioning and stabilising structure which is immediately superior to the ears of the patient, is positioned in a spaced relationship with respect to the ears of the patient 1000. For example, the region 8002 may be provided at least 5 mm above or superior to the ear of the patient 1000, such as at least 10 mm above or superior to the ear of the patient 1000. By positioning the region 8002 in a space relationship with respect to the ear of the patient (such as the tip of the ear of the patient), it may be possible to provide a positioning and stabilising structure with improved patient comfort.

A further feature of the design illustrated in FIG. 10B, is that the rigidiser 3340 is provided with a curvature that extends from a region superior to the ear of the patient 1000, through a midpoint 10004 which is substantially aligned with the Frankfort horizonal plane of the user and is anterior of the patient's ear. The rigidiser 3340 continues to curve such that the first end 5012 is inferior and anterior of the patient's ear, and optionally sits in a location which is more posterior than the midpoint 10004.

In some examples the first end 5012 of the rigidiser 3340 may overlie the jawbones of the patient in use.

This has the added effect of moving the pivot point 10002 for the headgear to a more anterior location on the patient's head than the example of FIG. 10A. For example, the pivot point 10002 may be substantially in line with the coronal plane of the patient in a location superior to the ear of the patient. In other examples the pivot points 10002 may be in a location which is superior to a centre of the ear of the patient.

The resulting pivot location is therefore more directly anterior to the first end 5012, and therefore the associated movement arc ‘A’ which the first end 5012 undergoes during the tightening of the headgear has a reduced movement component in the inferior to superior direction, and is predominantly in a posterior to anterior direction.

FIG. 10D shows an example of the rigidiser of FIG. 10C superimposed over part of a headgear in accordance with the present technology. In the illustrated example, the headgear comprises two rear straps 3307A, and 3307B, a top strap 3096 and at least one strap 3092 configured to connect to a patient interface (in some cases this may comprise a superior strap 3092 and an inferior strap 3094, as in the headgear described in, for example, FIG. 6G).

For sake of illustration, each of these straps are shown in part only to provide an example of the positioning of the rigidiser 3340 on or within the headgear. It should be appreciated that any one or more of these straps may extend further than shown, and may be provided with an adjustment mechanism as described herein.

FIG. 10E shows a top view of a patient's 1000 head with a rigidiser positioned against one side. From this view it can be seen that the rigidiser is configured to follow the contour of the patient's head in use. In other words, the side of the rigidiser facing the head of the patient has a slightly concave curvature. Accordingly, the shape of the rigidiser helps to improve the stability of the positioning and stabilising structure 3300 on the patient's head, and features described herein, such as having an extended third end 5010, can further assist with reducing the overall amount of movement experienced in use, for example by increasing the friction between the positioning and stabilising structure 3300 and the patient's head.

For example, the rigidiser 3340 in some examples may be formed to have a predetermined shape which is configured to match an average contour of the population. For example the rigidiser 3340 may be thermoformed together with one or more headgear straps in order to provide the predetermined shape.

### 5.3.4 Vent

In one form, the patient interface 3000 includes a vent 3400 constructed and arranged to allow for the washout of exhaled gases, e.g. carbon dioxide.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10 cmH2O, or at least 20 cmH2O.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

### 5.6 Glossary

For the purposes of the present technology disclosure, in certain forms of the present technology, one or more of the following definitions may apply. In other forms of the present technology, alternative definitions may apply.

### 5.6.1 General

Air: In certain forms of the present technology, air may be taken to mean atmospheric air, and in other forms of the present technology air may be taken to mean some other combination of breathable gases, e.g. oxygen enriched air.
Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.
Automatic Positive Airway Pressure (APAP) therapy: CPAP therapy in which the treatment pressure is automatically adjustable, e.g. from breath to breath, between minimum and maximum limits, depending on the presence or absence of indications of SDB events.
Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.
Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.
Flow therapy: Respiratory therapy comprising the delivery of a flow of air to an entrance to the airways at a controlled flow rate referred to as the treatment flow rate that is typically positive throughout the patient's breathing cycle.
Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.
Leak: The word leak will be taken to be an unintended flow of air. In one example, leak may occur as the result of an incomplete seal between a mask and a patient's face. In another example leak may occur in a swivel elbow to the ambient.
Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.
Noise, radiated (acoustic): Radiated noise in the present document refers to noise which is carried to the patient by the ambient air. In one form, radiated noise may be quantified by measuring sound power/pressure levels of the object in question according to ISO 3744.
Noise, vent (acoustic): Vent noise in the present document refers to noise which is generated by the flow of air through any vents such as vent holes of the patient interface.
Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.
Medical Oxygen: Medical oxygen is defined as oxygen enriched air with an oxygen concentration of 80% or greater.
Patient: A person, whether or not they are suffering from a respiratory condition.
Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar~0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.
Respiratory Pressure Therapy: The application of a supply of air to an entrance to the airways at a treatment pressure that is typically positive with respect to atmosphere.
Ventilator: A mechanical device that provides pressure support to a patient to perform some or all of the work of breathing.
Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.
Polycarbonate: a thermoplastic polymer of Bisphenol-A Carbonate.

### 5.6.1.1 Mechanics

Deformation: The process where the original geometry of a member changes when subjected to forces, e.g. a force in a direction with respect to an axis. The process may include stretching or compressing, bending and, twisting.
Elasticity: The ability of a material to return to its original geometry after deformation.
Resilient: Will release substantially all of the energy when unloaded. Includes e.g. certain silicones, and thermoplastic elastomers.
Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.
Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

### 5.6.2 Anatomy

### 5.6.2.1 Anatomy of the Face

Frankfort horizontal plane: A line extending from the most inferior point of the orbital margin to the left tragion. The tragion is the deepest point in the notch superior to the tragus of the auricle.
Otobasion inferior: The lowest point of attachment of the auricle to the skin of the face.
Otobasion superior: The highest point of attachment of the auricle to the skin of the face.

### 5.6.2.2 Anatomy of the Skull

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.
Parietal bones: The parietal bones are the bones that, when joined together, form the roof and sides of the cranium.
Temporal bones: The temporal bones are situated on the bases and sides of the skull, and support that part of the face known as the temple.

### 5.7 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1-25. (canceled)

26. A positioning and stabilising structure configured for use in supporting a patient interface in sealing engagement with the face of a patient during delivery of a flow of breathable gas to the nose and mouth of the patient during a respiratory therapy treatment, the positioning and stabilising structure comprising:
a rear strap configured to, in use, overlay the occipital bone and/or trapezius muscles at the rear of the patient's head;
a top strap, configured to, in use extend across the patient's head;
at least one side strap positioned on each side of the positioning and stabilising structure and being configured to connect to the patient interface in use, wherein each of the at least one side strap is configured to extend from the rear strap and top strap, at a point which is superior to a respective one of the patient's ears, through to the patient interface, along a respective side of the patient's face,
wherein each of the at least one side strap comprises a rigidiser, and wherein the rigidiser has a first end which overlays or sits inferior to a respective one of the patient's cheekbones in use, and a second end which is located posterior to a respective one of the patient's ears in use.

27. The positioning and stabilising structure of claim 26, comprising two side straps connecting to the patient interface to provide a two-point headgear arrangement.

28. The positioning and stabilising structure of claim 26, comprising four side straps connecting to the patient interface to provide a four-point headgear arrangement.

29. The positioning and stabilising structure of claim 28, wherein the four side straps comprise two superior side straps and two inferior side straps on each side of the patient's face, wherein the rigidiser is provided to at least each of the inferior side straps.

30. The positioning and stabilising structure of claim 26, wherein an axis connecting the first end of the rigidiser to the second end of the rigidiser has an angle when measured with respect to the Frankfort horizontal of the patient in use, and wherein the angle is between substantially 0 degrees and substantially 25 degrees.

31. The positioning and stabilising structure of claim 30, wherein the angle is substantially 15 degrees.

32. The positioning and stabilising structure of claim 26, wherein the rigidiser has a profile which follows the direction of the at least one side strap.

33. The positioning and stabilising structure of claim 32, wherein the profile comprises a substantially inverted U-shape.

34. The positioning and stabilising structure of claim 32, wherein the profile comprises a substantially inverted Y-shape.

35. The positioning and stabilising structure of claim 26, wherein the rigidiser is configured to extend in the direction of the top strap, from a region superior to the ear of the patient.

36. The positioning and stabilising structure of claim 26, wherein the rigidiser is internal to the at least one side strap.

37. The positioning and stabilising structure of claim 26, wherein the rigidiser is external to the at least one side strap.

38. The positioning and stabilising structure of claim 26, wherein the top strap is configured to, in use overlay the parietal bone on the top of the patients head.

39. The positioning and stabilising structure of claim 26, wherein the rigidiser comprises a first limb and a second limb, the first and second limb being offset laterally from a longitudinal axis of the at least one side strap.

40. The positioning and stabilising structure of claim 39, where the first limb and second limb are spaced apart to provide a gap between the first limb and the second limb.

41. The positioning and stabilising structure of claim 39, wherein a first end of the first limb is connected to a first end of the second limb, and a second end of the first limb is connected to a second end of the second limb.

42. The positioning and stabilising structure of claim 41, wherein a first end of the first limb is connected to a first end of the second limb, and a second end of the first limb, is separated from the second end of the second limb.

43. A respiratory therapy system, comprising:
a patient interface configured to provide a seal with the airways of a patient for delivery of a flow of breathable gas at a positive pressure; and
the positioning and stabilising structure of claim 26.

44. The respiratory therapy system of claim 43, wherein the patient interface is configured to provide the flow of breathable gas to the oral and nasal passages of the patient.
